(19) United States
(12) Patent Application Publication
(10) Pub. No.: US 2014/0072801 A1
(43) Pub. Date: Mar. 13, 2014

(54) PEPTIDE NANOTUBE DEVICE AND MANUFACTURING METHOD THEREOF

(71) Applicants: Jiahorng Liaw, Taipei City (TW); Wei-Hsien Hsieh, Taipei City (TW); Jeng-Hsien Chen, Taipei City (TW); Shwu-Fen Chang, Taipei City (TW); Hui-Min Chen, Taipei City (TW)

(72) Inventors: Jiahorng Liaw, Taipei City (TW); Wei-Hsien Hsieh, Taipei City (TW); Jeng-Hsien Chen, Taipei City (TW); Shwu-Fen Chang, Taipei City (TW); Hui-Min Chen, Taipei City (TW)

(73) Assignee: Jiahorng Liaw, Taipei City (TW)

(21) Appl. No.: 13/612,437

(22) Filed: Sep. 12, 2012

(51) Int. Cl. C07K 14/00; B82Y 40/00; B82Y 30/00

(52) U.S. Cl. 428/401; 530/409; 977/795

(57) ABSTRACT

A peptide nanotube (PNT) device and method of manufacturing thereof are disclosed herein. The PNT device comprises PNTs composed of cyclo-(D-Trp-Tyr) peptide and a matrix, including biomolecules, complexed with the PNTs. The PNT device is biodegradable and biocompatible, as well as capable of being uptake by mammalian cells. Wherein, the biomolecules comprise peptides, proteins, nucleic acids including DNA, shRNA and siRNA, and drugs. The method for manufacturing PNT device comprises: dissolving a cyclo-(D-Trp-Tyr) peptide powder in a solvent to be a solution in a container; incubating the solution at a predetermined temperature for a predetermined time for the solvent to evaporate to obtain PNTs formed of cyclo-(D-Trp-Tyr) peptide; and mixing the PNTs with a matrix including biomolecules to obtain the PNT device.

### BACKGROUND

1. Field of the Invention

The present invention generally relates to a nanotube device and manufacturing method thereof, in particular to a peptide nanotube device and a manufacturing method thereof.

2. Description of the Related Art

There is enormous interest in novel nanocarriers for biomedical usage. For example, it was reported that microspheres formed by elastin-like polypeptide (ELP) and micelles formed by amphiphilic elastin-mimetic recombinant protein were capable of solubilizing, encapsulating, and controlling drug release of hydrophobic drug.¹ In addition, ELP-modified liposome was more easily internalized into HeLa cells than unmodified liposome.² Furthermore, plasmid DNA formulated by flexible poly(ethylene oxide)-polypropylene oxide)-poly(ethylene oxide) polymeric micelles could enhance transdermal permeability in vitro and gene expression in vivo.³

Recently, high aspect ratio (AR) particles, such as nanotubes, draw attentions because of their bulk capability,⁴ faster internalization rates, larger amounts of internalization, as well as prolonged blood circulation time than their spherical counterparts.⁵ In addition, different functionalized surfaces of carbon nanotubes (CNTs), such as the addition of poly(vinyl pyrrolidone) and poly(styrene sulfonate),⁶ have been proven to enhance the solubility of CNTs in aqueous conditions. Moreover, peptide-functionalized carbon nanotubes were reported to have a higher uptake in living cells.⁷ However, the toxicity of CNTs, such as the induction of cell apoptosis,⁸ epithelial granuloma, interstitial and peribronchial inflammation, necrosis,⁹ and lung fibrosis¹⁰ by a single intratracheal instillation of CNTs, as well as hepatotoxicity by intravenous injection¹¹, have the drawback for the clinical utilization of CNTs.

It is known in the art that surface properties, such as hydrophobicity, size, radius of curvature, charge, and coatings generating steric or electrosteric effects of nanocarriers, influence the interaction of nanocarriers with cell membranes and entry of the nanocarriers to cells. It was suggested that the internalization of CNTs in HL60 cells was initiated by nonspecific association of the hydrophobic regions in nanotubes with cell membranes.¹² CNTs were taken up by HepG2 cells through a size-dependent and energy-dependent endocytosis.¹³ In addition, polystyrene particles with a prolate ellipsoid geometry were found to be phagocytized faster when the particles with high radius of curvature area contacted with the macrophage cell.¹⁴ Moreover, nanorods with a cationic charge by coating with poly(diallyldimethyl ammonium chloride) or cetyltrimethylammonium bromide were much easier to be internalized by MCF-7 cells than that with a anionic charge through coating with poly(styrene sulfonate),¹⁵ CNTs with steric hindrance generated by modifying with poly(ethylene glycol) (PEG) polymers had a longer half-life in blood circulation, reduced reticuloendothelial system (RES) uptake, and reduced toxicity.¹⁶

On the other hand, oral biomolecule delivery is attractive due to factors such as ease of administration, leading to improved patient convenience and compliance, thereby reducing overall healthcare costs. However, effective oral biomolecule administration is desirable but quite challenging owing to the nature of the gastrointestinal (GI) tract. The extremely acidic pH in the stomach and the presence of enzymes may cause biomolecule degradation. Secreted pancreatic enzymes in the lumen of the intestine may also cause substantial loss of biomolecule activity. Finally, the physical barrier of the intestinal cells must be crossed before a biomolecule reaches the circulation. For macromolecular biomolecules, it may be especially problematic for too large to pass through cells. These obstacles lead to poor oral bioavailability for many biomolecules.

In addition, nucleic acids are increasingly being applied as drugs, either as components of a vaccine or in gene therapy approaches. Except the above-mentioned barriers for decreasing the bioavailability of biomolecules, additional barriers may exist to affect the efficiency of nucleic acid delivery, e.g. endosomal escape, nuclear localization, transcription, translation, protein processing, and protein secretion into plasma.

Consequently, a conventional method for oral biomolecule delivery or other delivery routes may have the problems of incapability of penetrating biomembranes, instability of biomolecules in GI tract and low bioavailability of biomolecules to the desired tissues or organs. Therefore, the inventor of the present invention designs a peptide nanotube device and manufacturing method thereof to improve the conventional flaws to further increase the implementation and utilization in industries.

### BRIEF SUMMARY

Therefore, it is a primary objective of the present invention to provide a peptide nanotube (PNT) device and a manufacturing method thereof to achieve the effect of increasing the bioavailability of the biomolecules to the desired tissues or organs.

To achieve the foregoing objective, the present invention provides a peptide nanotube device, and the PNT device comprises PNTs composed of cyclo-(D-Trp-Tyr) peptide and a matrix, including biomolecules, complexed with the PNTs.

Preferably, the PNT device is biodegradable and biocompatible.

Preferably, the PNT device capable of being uptake by mammalian cells.

Preferably, the matrix is complexed inside the PNTs or on a surface of the PNTs.

Preferably, the PNT device is stable under gastric acid, bile and deoxyribonuclease.

Preferably, the biomolecules comprise peptides, proteins, nucleic acids and drugs.

Preferably, the nucleic acids comprise DNA, shRNA and siRNA.

Preferably, a release rate of the PNT device to release DNA is about 1×10¹⁰ to 5×10¹¹ copies DNA/t^{1/2}.

Preferably, wherein a width of the PNTs is 10 to 800 nm.

Preferably, wherein a length of the PNTs is 0.1 to 20 um.

Preferably, wherein the PNTs is single nanotube bundled or aggregated.

Preferably, a zeta potential of the PNT device is −10 to 10 mV.

To achieve the foregoing objective, the present invention further provides a method for manufacturing peptide nanotube (PNT) device, the method comprises dissolving a cyclo-(D-Trp-Tyr) peptide powder in a solvent to be a solution in a container; incubating the solution at a predetermined temperature for a predetermined time for the solvent to evaporate to obtain a peptide nanotubes formed of cyclo-(D-Trp-Tyr) peptide; and mixing the peptide nanotubes with a matrix including biomolecules to obtain the PNT device.

Preferably, the solvent is trifluoroacetic acid.

Preferably, a concentration of the trifluoroacetic acid is 0.1 to 5%.

Preferably, a volume of the trifluoroacetic acid is 0.015 to 0.75 mL.

Preferably, a weight of the cyclo-(D-Trp-Tyr) peptide powder is 0.1 to 10 mg.

Preferably, after dissolving the cyclo-(D-Trp-Tyr) peptide powder in the solvent, the method further comprise the step of adding double distilled water to the solution at the predetermined temperature.

Preferably, the predetermined temperature is 0-25° C.

Preferably, the predetermined time is 10-72 hours.

Preferably, the solvent is ethanol.

Preferably, a concentration of the ethanol is 1 to 100%.

Preferably, a volume of the ethanol is 0.1 to 10 mL.

Preferably, a weight of the cyclo-(D-Trp-Tyr) peptide powder is 0.1 to 10 mg.

Preferably, the predetermined temperature is 0-25° C.

Preferably, the predetermined time is 1-48 hours.

Preferably, the biomolecules comprise peptides, proteins, nucleic acids and drugs.

Preferably, the nucleic acids comprise DNA, shRNA and siRNA.

Preferably, a concentration of the DNA is 0.01-0.3 μg/μL.

Preferably, a concentration of the peptide nanotubes is 0.05-5% (w/v).

The PNT device and the manufacturing method thereof according to the present invention adopt a detachable structure, so that the present invention has the following advantages:

(1) The peptide nanotube device and the manufacturing method thereof of the present invention can be used to deliver biomolecules orally or other delivery routes to the living organism with high efficiency.

(2) The peptide nanotube device and the manufacturing method thereof of the present invention may increase the capability of the peptide nanotube device to penetrate the biomembranes, and thereby increasing the bioavailability of the biomolecules to the desired tissues or organs.

(3) The peptide nanotube device and the manufacturing method thereof of the present invention may enhance the stability of the biomolecules throughout the biomembranes administration so as to increase the efficacy of the biomolecules.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The properties and effects of the present invention will now be described in more details hereinafter with reference to the accompanying drawings that show various embodiments of the invention as follows.

FIG. 1 is a view of peptide nanotube devices prepared with trifluoroacetic acid (TFA) in accordance with an embodiment of the present invention. Wherein, part A is an optical microscope view of peptide nanotube devices prepared with TFA; parts B-E are scanning electron microscope (SEM) views of peptide nanotube devices prepared with TFA; parts F and G are transmission electron microscope (TEM) views of peptide nanotube devices prepared with TFA; and parts H and I are an atomic force microscope (AFM) view and a cross section of AFM image along the line in part H, respectively, of peptide nanotube devices prepared with TFA.

FIG. 2 is a schematic view of self-association and size distribution of peptide nanotube devices prepared with TFA in accordance with an embodiment of the present invention.

FIG. 3 is a schematic view of either pCMV-lacZ plasmid or pCMV-lacZ plasmid formulated with peptide nanotube devices prepared with TFA in accordance with an embodiment of the present invention. Wherein, part A is an AFM view of pCMV-lacZ plasmid alone; part B is a SEM view of pCMV-lacZ plasmid formulated with peptide nanotube devices prepared with TFA; and parts C and D are an AFM view and a cross section of AFM image along the line in part C, respectively, of pCMV-lacZ plasmid formulated with peptide nanotube devices prepared with TFA.

FIG. 4 is a fluorescence microscope view of TM-rhodamine labeled pCMV-lacZ plasmid formulated with peptide nanotube devices prepared with TFA in accordance with an embodiment of the present invention.

FIG. 5 is a schematic view of pCMV-lacZ plasmid formulated with peptide nanotube devices prepared with TFA in accordance with an embodiment of the present invention. Wherein, parts A-C are emission fluorescence spectra of pCMV-lacZ plasmid formulated with peptide nanotube devices prepared with TFA; and part D is a linear plot of pCMV-lacZ plasmid formulated with peptide nanotube devices prepared with TFA.

FIG. 6 is a schematic view of characterization of pCMV-lacZ plasmid formulated with peptide nanotube devices prepared with TFA in accordance with an embodiment of the present invention. Wherein, part A is a schematic view of qPCR quantification analysis of the amount of pCMV-lacZ absorbed in peptide nanotube devices prepared with TFA; and part B is a schematic view of pCMV-lacZ release profile in peptide nanotube devices prepared with TFA.

FIG. 7 is a schematic view of the stability of pCMV-lacZ formulated with peptide nanotube devices prepared with TFA with treatment of DNase I (part A), gastric acid (part B) and bile (part C), respectively, in accordance with an embodiment of the present invention.

FIG. 8 is a schematic view of the stability of the peptide nanotube devices prepared with TFA with treatment of gastric acid in accordance with an embodiment of the present invention. Wherein, part A is fluorescence microscope (top panel) or bright field (BF) views (bottom panel) of the stability of the peptide nanotube devices prepared with TFA with treatment of gastric acid for indicated time interval; and part B is an AFM view of the stability of peptide nanotube devices prepared with TFA with treatment of gastric acid.

FIG. 9 is histological views of X-Gal staining of the various tissues of nude mice with oral delivery of pCMV-lacZ formulated with peptide nanotube devices prepared with TFA in accordance with an embodiment of the present invention.

FIG. 10 is fluorescence microscope views of the various tissues of nude mice with oral delivery of TM-rhodamine labeled pCMV-lacZ formulated with peptide nanotube devices prepared with TFA in accordance with an embodiment of the present invention.

FIG. 11 is southern blot analysis of the tissue DNAs from various tissues of mice with oral delivery of naked pCMV-lacZ or pCMV-lacZ formulated with peptide nanotube devices prepared with TFA, respectively, in accordance with an embodiment of the present invention.

FIG. 12 is an ex vivo bioluminescence view of the various tissues of mice with oral delivery of pCMV-hRluc formulated with peptide nanotube devices prepared with TFA in accordance with an embodiment of the present invention.

FIG. 13 is immunohistological analysis of the various tissues of mice with oral delivery of pCMV-hRluc formulated with peptide nanotube devices prepared with TFA in accordance with an embodiment of the present invention.

FIG. 14 is thioflavin T (ThT) image merged with DAPI image, bright field image, and ThT image merged with DAPI image and bright field image, respectively, of histological analysis of the various tissues of mice with oral delivery of peptide nanotube devices prepared with TFA in accordance with an embodiment of the present invention.

FIG. 15 is a schematic view of peptide nanotube devices prepared with ethanol in accordance with an embodiment of the present invention. Wherein, parts A and B are scanning electron microscope (SEM) views of peptide nanotube devices prepared with ethanol; parts C and D are an atomic force microscope (AFM) view and a cross section of AFM image along the line in part C, respectively, of peptide nanotube devices prepared with ethanol; and part E is a fluorescence microscope view of peptide nanotube devices prepared with ethanol.

FIG. 16 is a schematic view of self-association of peptide nanotube devices prepared with ethanol in accordance with an embodiment of the present invention.

FIG. 17 is immunohistological analysis of liver tissues (part A) and lung tissues (part B), respectively, of mice with oral delivery of pCMV-hRluc formulated with peptide nanotube devices prepared with ethanol in accordance with an embodiment of the present invention.

FIG. 18 is histological analysis of the brain tissues (part A) and lung tissues (part B) of mice with orally delivery of ThT pre-stained peptide nanotube devices prepared with ethanol in accordance with an embodiment of the present invention.

FIG. 19 is ThT images merged with DAPI image and bright field image of histological analysis of the cornea tissues of mice with topically eye drop delivery of ThT pre-stained peptide nanotube devices prepared with ethanol in accordance with an embodiment of the present invention. Wherein, parts A and B is merged images of histological analysis of the epithelial layers of the cornea tissues of mice with topically eye drop delivery of ThT pre-stained peptide nanotube devices prepared with ethanol; part C is a merged image of histological analysis of the stroma layers of the cornea tissues of mice with topically eye drop delivery of ThT pre-stained peptide nanotube devices prepared with ethanol.

### BRIEF DESCRIPTION OF THE SEQUENCES

SEQ ID NO: 1 is a qPCR forward primer for 13-Gal.

SEQ ID NO: 2 is a qPCR reverse primer for B-Gal.

SEQ ID NO: 3 is a PCR forward primer for Renilla luciferase.

SEQ ID NO: 4 qPCR reverse primer for Renilla luciferase.

### DETAILED DESCRIPTION

The technical content of the present invention will become apparent by the detailed description of the following embodiments and the illustration of related drawings as follows. The disclosure of “Oral Gene Delivery with cyclo-(D-Trp-Tyr) Peptide Nanotubes”, published on Mol. Pharmaceutics, 2012, 9 (5), pp 1231-1249, is incorporated herein in its entirety by reference.

### Material and Methods

### Preparation of cyclo-(D-Trp-Tyr) Peptide Nanotubes (PNTs) with trifluoroacetic acid (TFA)

The self-assembly of cyclo-(D-Trp-Tyr) peptide (Bachem, Bubendorf, Switzerland) PNTs was prepared as the following description. Briefly, 0.1 to 10 mg of cyclo-(D-Trp-Tyr) peptide powder may be used to be dissolved in 0.015 to 0.75 mL of trifluoroacetic acid (TFA). Wherein, the concentration of the TFA may be 0.1 to 5%, preferably 3.5%. In the preferred embodiment, 5 mg of cyclo-(D-Trp-Tyr) peptide powder may be dissolved in 0.5 mL of trifluoroacetic acid (TFA) in an Eppendorf tube. Then, double distilled water was added into the Eppendorf tube at 0 to 25° C. The Eppendorf tube was left opened and then floated in an airtight vial which was filled with 15 mL of double-distilled water. The white suspension of nanotube may be obtained after incubation for 10 to 72 h at 0 to 25° C. Preferably, the white suspension of nanotube may be obtained after incubation for 24 h at 25° C. PNTs were harvested by centrifugation and washed repeatedly with double-distilled water to remove the residual TFA. Specifically, the amount of cyclo-(D-Trp-Tyr) peptide powder and TFA may be varied and should not be construed as limited to the embodiments set forth herein.

### Preparation of cyclo-(D-Trp-Tyr) Peptide Nanotubes (PNTs) with ethanol

The self-assembly of cyclo-(D-Trp-Tyr) peptide (Bachem, Bubendorf, Switzerland) PNTs was prepared as the following description. Briefly, 0.1 to 10 mg of cyclo-(D-Trp-Tyr) peptide powder may be used to be dissolved in 0.1 to 10 mL of ethanol. Wherein, the concentration of the ethanol may be 1 to 100%, preferably 50%. In the preferred embodiment, 5 mg of cyclo-(D-Trp-Tyr) peptide powder may be dissolved in 10 mL of ethanol in a beaker and The white suspension of nanotube may be obtained after incubation for 1 to 48 h at 0 to 25° C. Preferably, the white nanotube may be obtained after incubation for 24 h at 25° C. PNTs were harvested after ethanol and water evaporation. Specifically, the amount of cyclo-(D-Trp-Tyr) peptide powder and ethanol may be varied and should not be construed as limited to the embodiments set forth herein.

### Plasmid DNA

pCMV-lacZ and pCMV-hRluc plasmids, carrying the lacZ gene encoding β-Gal and hRluc gene encoding humanized Renilla reniformis luciferase, respectively, under the control of the cytomegalovirus (CMV) promoter, were the transferred DNA in the present invention. These plasmids were amplified in the Escherichia coli host strain DH5α and purified by equilibrium centrifugation on a CsCl-ethidium bromide gradient. The purity of the plasmid DNA prepared was determined by electrophoresis on an agarose gel followed by ethidium bromide staining. DNA concentration was measured by ultraviolet (UV) absorption at 260 nm.

### Plasmid DNA Labeling

Plasmid DNA, pCMV-lacZ, was labeled with TM-rhodamine (Lable IT nucleic acid labeling kit; Mirus, Madison, Wis.) according to the manufacturer's and labeling reagent. Briefly, pCMV-lacZ was mixed with labeling buffer and labeling reagent. After incubating at 37° C. for 2 h, the labeled DNA was further purified by ethanol precipitation and confirmed by HPLC with TSK-GEL G5000 PWXL column (Tosoh Bioscience, Tessenderlo, Belgium) under a 0.7 mL/min flow rate of water (pH 5) mobile phase and fluorescence detector (excitation: 546 nm; emission: 576 nm).

### Formulation of Biomolecule/PNT Complexes

The prepared may be used to formulate with various biomolecules, such as peptides, proteins, nucleic acids, drugs and the like. Wherein, the nucleic acid may comprise DNA, shRNA and siRNA. In an embodiment, PNTs may be formulated with different kinds of plasmid, e.g. pCMV-lacZ and pCMV-hRluc. The concentration of the plasmid to be formulated with the PNTs may be 0.01 to 0.3 μg/μL, wherein the concentration of the PNTs may be 0.05 to 5% (w/v). In a preferred embodiment, the pCMV-lacZ/PNT or TM-rhodamine-labeled pCMV-lacZ/PNT or pCMV-hRluc/PNT complexes were formulated by gently mixing plasmid DNA (0.26 μg/μL) with PNTs (0.15%, w/v) in an Eppendorf tube for 1 to 24 h at 4 to 37° C. and should not be construed as limited to the embodiments set forth herein.

Characterization of pCMV-lacZ/PNTs

### 1. Scanning Electron Microscope (SEM) Imaging

The PNT suspension was dropped on the mica surface and dried in a vacuum system. Samples were then coated with gold particles using a sputter coating method under vacuum of 2 mbar at 20 mA for 8 min and further observed by SEM. SEM (S-2400/Hitachi Instruments Inc., San Jose, Calif.) was operated at an accelerating voltage of 15 kV and 20 kV.

### 2. Transmission Electron Microscope (TEM) Imaging

PNTs were dried under vacuum system and then embedded in epoxy resin and followed by thin section preparation. Sample films with an 80 nm thickness were picked up on 200 mesh carbon-coated copper grid for TEM imaging. Bright-field TEM imagings of the PNTs were performed on a TEM (H-600, Hitachi Instruments Inc., San Jose, Calif.) operating at 80 kV. Images were taken under 40000× zoom field.

### 3. Atomic Force Microscope (AFM) Imaging

A 10 μL PNT suspension was placed on a mica surface without further treatment. The AFM (diCPII; Digital Instruments/Veeco Metrology Group, Santa Barbara, Calif.) was operated in a constant tapping mode. The cantilevers were standard NanoProbe silicon single crystal lever (NSC15/AIBS; MikroMasch, Estonia). The constant force mode was used with a recommended scan frequency of 328 kHz. A scanner with a 2 μm scanning range was used, and all images were collected within a 4 μm² square area.

### 4. Fluorescence Microscope Imaging

A sample of 10 μL of TM-rhodamine labeled pCMV-lacZ/PNT complexes were placed on a slide surface and air-dried. The labeled and without labeled groups were fixed exposure times imaged by a fluorescence microscope (Olympus BX40, Japan).

### 5. Critical Association Concentration (CAC) Measurements

The association of PNTs was evaluated using pyrene as a fluorescence probe. The fluorescence spectrum of pyrene in the PNT solution was measured using a Hitachi F-4500 fluorescence spectrophotometer (Hitachi, Tokyo, Japan). The emission spectrum scan was performed from 350 to 460 nm using a fixed excitation wavelength of 339 nm with a constant pyrene concentration of 6×10⁻⁷ M. The PNT concentrations were from 1.6 μg/mL to 1.6 mg/mL. CAC was determined by the pyrene I₁/I₃ ratio method. The ratio of the fluorescence intensity at 373 nm (I₁) and at 383 nm (I₃) was plotted against the logarithm of the concentration of associating molecules. The CAC value was determined from the crossover point of the rapidly varying part and the nearly horizontal part at low concentrations.

### 6. Size and Zeta Potential Measurements

The size of PNT suspensions at various concentrations and the zeta potential of pCMV-lacZ, PNTs alone, and pCMV-lacZ/PNT complexes in water were measured by quasielastic laser dynamic light scattering (DLS) (Hydro 2000S and nano series nano-ZS, respectively; Malvern Instruments, Malvern, U.K.) as incorporated by reference 17. All measurements were performed at 25° C. at a measurement angle of 90° with an assumed refractive index ratio of 1.33.

### 7. Fluorescence Measurements

To determine the association constant of the binding of Tyr in PNTs and the plasmid DNA, fluorescence measurements were performed. The emission spectra (emission slit 2.5 nm, F-4500 spectrophotometer, Hitachi Instruments Inc., Tokyo, Japan) were measured upon excitation at 280 nm (excitation slit 2.5 nm), where both of Trp and Tyr residues were excited and at 295 nm where only Trp residues were selectively excited. The binding constant K of Tyr to DNA was evaluated by the change of intensity in fluorescence emission spectra of PNTs in the presence of different concentrations of DNA excitation at 280 nm and according to equation 1 as incorporated by reference 18 and 19.

$$log [ \frac{{F}_{0} - F}{F} ] = log K + n log [ DNA ]$$

Here F₀ and F are the fluorescence intensity from the fluorophore, Tyr, at 280 nm in the absence and the presence of different concentrations of DNA, respectively.

8. Loading Efficiency of pCMV-lacZ/PNTs

An amount of 40 μg of pCMV-lacZ was added to 2-fold serial dilutions of PNT suspension and incubated for 24 h at ambient conditions, respectively. The pCMV-lacZ/PNT complexes were centrifuged at 16000 g for 10 min at 25° C., and the precipitates were collected for further phenol-CIAA (chloroform/isoamyl alcohol=1:1; v/v) extraction. After extraction, the DNA pellets were dissolved in water and quantified by PCR (qPCR). qPCR was performed using a SYBR Green PCR Master Mix in an ABI PRISM 7300 sequence detection system (Applied Biosystems, 7300 System Sequence Detection System (SDS) software, version 1.3). The primers for β-Gal (forward: 5′-CTA CAC CAA CGT AAC CTA TCC C-3′ (SEQ ID NO: 1) and reverse: 5′-TTC TCC GGC GCG TAA AAA TGC G-3′ (SEQ ID NO: 2)) were used. The conditions for the PCR were as follows: 50° C. for 2 min, 95° C. for 10 min, and 40 cycles of 95° C. for 15 sec and 60° C. for 1 min. All samples were run in duplicate with a set of plasmid standards that contained 1×10² to 1×10⁸ copies of the lacZ gene. The quantification values were obtained from the threshold cycle (Ct) number at which the increase in signal associated with an exponential growth of PCR products began to be detected using SDS software.

### 9. In Vitro Membrane Release of Plasmid DNA

To observe the effect of PNTs on plasmid release, a Franz cell with a 0.2 μm membrane disk filter (Supor-200, PALL Life Sciences, Ann Arbor, Mich., USA) was used for the in vitro release study. An active diffusion area of 0.63 cm² was exposed to the donor and receiver compartments of Franz cell, containing 6 mL of phosphate buffer solution (PBS; pH 7.4) in receiver site. An amount of 490 μL of naked pCMV-lacZ (0.26 μg/μL) or pCMV-lacZ formulated with PNTs (1.5 mg/mL) was added to the donor compartment, and 0.2 mL samples were taken from the receiver compartment at designed sampling times; the volume in the receiver compartment was maintained by the addition of 0.2 mL of pre-warmed PBS. Samples were quantified by qPCR same as described in the loading efficiency of pCMV-lacZ/PNTs section (Section 8). The release time profile of DNA was obtained by plotting the cumulative amount of DNA released against time.

Stability of pCMV-lacZ/PNTs with DNase I, Simulated Gastric Acid, or Bile Digestion

The protection of pCMV-lacZ with PNTs against DNase I was carried out as described in the following procedure. Briefly, the mixtures of 13 units of RQ1 RNase-free DNase I (Promega Biotech Co., Ltd., Madison, Wis.) and 100 μg of pCMV-lacZ with or without PNTs in a total volume of 200 μL were incubated at 37° C. The mixtures were sampled with 10 μL each after incubating with DNase I at 37° C. for 0, 5, 10, 15, 20, 30, 40, 50, 60, 70, 80, 100, and 120 min, and then 1 μL of RQ1 DNase I stop solution (Promega Biotech Co., Ltd., Madison, Wis.) was immediately added into each sample. The stability of pCMV-lacZ/PNT formulation with simulated gastric acid was performed as following. Briefly, pCMV-lacZ solution with or without PNTs was adjusted to pH 2 with simulated gastric acid. After incubating at 37° C. for 0, 30, 60, 90, 120, 180, 240, 300, and 360 min, the 10 μL samples collected at indicated time points were neutralized with 25 mM ethylenediaminetetraacetic acid (EDTA) solution (pH 8). The stability of pCMV-lacZ with PNTs against bile was carried out as following description. Bile, isolated from mice bile duct, was added to pCMV-lacZ or pCMV-lacZ/PNTs solution at a final concentration of 10% (v/v) and incubated at 37° C. At 0, 10, 20, 30, 40, 60, 90, 120, 180, 240, 300, and 360 min time points, each 10 μL of samples were mixed with 25 mM EDTA solution (pH 8). The resulting solutions were directly loaded onto a 0.8% agarose gel for electrophoresis, and then the gel was stained with ethidium bromide. The qualification of band intensities was performed with a Kodak EDAS290 analysis system (Kodak Scientific Imaging System, New Haven, Conn.).

Stability of PNTs with Simulated Gastric Acid Treatment

Since there are no available methods to determine the in vivo fate of PNT after oral delivery, the inventors have mimicked the in vivo situation and analyzed the degradation of PNTs in the presence of simulated gastric acid (pH 2). Briefly, 0.2 mg of PNTs pre-stained with thioflavin T (4 μM), a dye that has been used to stain PNTs, for 5 min was incubated with 150 μL of simulated gastric acid for 0, 20, 40, 60, 80, and 100 min. The morphological change of thioflavin T pre-stained PNTs at different time points of treatment was analyzed with fluorescence microscopy (Olympus BX40, Japan) and on a mica surface observed with AFM as previous section.

### Animals

The animal protocol was approved by the Laboratory Animal Research Committee of Taipei Medical University. Male nude mice (BALB/cAnN-Foxn1nu/CrlNarl) at 6-8-week age were used for in vitro duodenal penetration and in vivo oral delivery studies and were purchased from the National Laboratory Animal Breeding and Research Center (Taipei, Taiwan). They were maintained under specific pathogen-free conditions.

### In Vitro Duodenal Penetration Studies.

For the in vitro DNA permeation studies, nude mice were sacrificed by cervical dislocation and upper duodenal sections, from the pylorus to 1 cm distal to the ligament of Treitz, were retrieved. Duodenal tissues were gently rinsed three times in 4 or 37° C. phosphate buffered saline (PBS) or pretreated with PBS containing 150 mM of sodium azide for 15 min and then placed in an in vitro vertical diffusion apparatus. A tissue surface area of 0.13 cm² was exposed to the donor and receiver compartments of Franz cell, containing 3 mL of PBS in receiver site. An amount of 150 μL of naked DNA (0.26 μg/μL) or DNA formulated with four different concentrations of PNTs (0.01, 0.2, 0.8, and 1.5 mg/mL) was added to the donor compartment, and an aliquot of 0.2 mL sample was taken from the receiver compartment at indicated sampling times; the volume in the receiver compartment was maintained by the addition of 0.2 mL of pre-warmed PBS. Samples were then followed by the phenol-CIAA extraction and ethanol precipitation. The purified DNA was re-dissolved in TE buffer, and the concentration was quantified by qPCR, the same as described in the loading efficiency of pCMV-lacZ/PNTs section (section 8). The apparent permeability coefficient (Pₐₚₚ) was calculated according to the following equation: Pₐₚₚ=(dC/dt) V/A×C₀, where V(dC/dt) is the steady state rate of DNA appearing in the receiver chamber after the initial lag time, C₀ is the initial plasmid concentration in the donor chamber, and A is the area of duodenal tissue exposed (0.13 cm²). Data from all experiments were pooled to determine the mean and standard error. The analysis of variance (ANOVA) using Dunnett's multiple comparison tests with a 95% confidence level determined the significance of differences between each group of experiments.

### Oral Gene Transfer In Vivo

For the in vivo studies, nude mice were fasted but allowed free access to water for 24 h before the experiments. Formulations (pCMV-lacZ/PNTs or pCMV-hRluc/PNTs) were administered with a stomach feeding needle for mice (KN-342; Natume Seisakusho). Eight doses of formulated complexes (150 μL), containing plasmid (0.26 μg/μL) and PNTs nanotubes (1.5 mg/mL), were administrated at 3 h intervals (9 a.m., 12 a.m., 3 p.m., and 6 p.m.). Mice receiving only plasmid DNA served as control groups. To evaluate gene transfer in vivo, mice were sacrificed by cervical dislocation at 48 and 72 h after the first dose and all organs and tissues including the duodenum, testis, kidney, stomach, heart, liver, brain, lung, spinal cord, and spleen were removed and processed immediately for individual analysis.

### Preparation of Tissue Extracts and Determination of Transgene Expression

The β-Gal expression was quantified with the enzyme substrate chlorophenol red-β-D-galactopyranoside (CPRG; Gene Therapy Systems, San Diego, Calif.). Color development was measured at 580 nm. For Renilla luciferase activity measurement, tissues were lysed and mixed with luciferase substrate using a Renilla luciferase assay kit (Promega, Madison, Wis.). The luciferase activity was measured in a photoluminometer (Thermo Varioskan Flash, Thermo Scientific, CA) over 10 sec and was calculated as the number of relative light units (RLU). Total tissue proteins were measured with a DC protein assay reagent kit (Bio-Rad, Hercules, Calif.) and used to normalize the β-Gal and Renilla luciferase activity for each sample. Statistical comparisons were determined by ANOVA (Dunnett's multiple comparison tests) with a 95% confidence level.

Tissue Section for pCMV-lacZ Delivery and pCMV-hRluc Delivery

Animal tissues were first washed with ice-cold PBS solution and immersed in fixation solution (4% paraformaldehyde) for 1.5 h at 4° C. Tissues were then stained with X-gal solution at 37° C. for 2 days and further dehydrated in 40% sucrose solution for 12 h. Cryosections (10 μm) of the O.C.T.-embedded tissues were fixed with acetone/methanol (1:1) on ice for 10 min. For pCMV-lacZ delivery, the additions of EGTA and Mg ion, as well as the reaction at high pH conditions, were applied in this assay to reduce the endogenous β-Gal activity. After hematoxylin and eosin (HE) staining, the slides were sealed with Leica CV Mount. The sections were observed using optical microscope (Olympus BX40, Japan). For pCMV-hRluc delivery, the sections were blocked by 1% bovine serum albumin (BSA) for 30 min at room temperature. The cryosection was hybridized with rabbit anti-Renilla luciferase antibody (1:100, MBL International Corporation, Woburn, Mass.), incubated in moisture conditions at 4° C. overnight, and then washed by PBS and hybridized with donkey anti-rabbit IgG-FITC (1:100, Santa Cruz Biotechnology Inc., Santa Cruz, Calif.) in the dark for 1 h at room temperature. The section was washed with PBS, stained with propidium iodide (PI, 40 ng/mL, Roche Diagnostic Corp., Indianapolis, Ind.) for the localization of the nucleus, and then sealed with Leica CV Mount. The control and experimental groups were observed by a fluorescence microscope (Olympus BX40, Japan) with FITC and PI filter at the fixed exposure time.

Distribution of pCMV-lacZ In Vivo

To trace the distribution of delivered DNA, the complexes of TM-rhodamine labeled pCMV-lacZ/PNTs were administrated following methods described in the oral gene transfer in vivo section. Mice receiving no treatment served as control groups. Mice were sacrificed by cervical dislocation at 1 h after the first dose and β-Gal expressing tissues including the stomach, duodenum, liver, and kidney were removed and immersed in fixation solution (4% paraformaldehyde, Merck, Darimstadt, Germany) for 24 h. After dehydration with the concentration gradient of ethanol (70%, 80%, 95%, and 100%), tissues were embedded into paraffin blocks. After de-paraffinization, re-hydration, and DAPI (1 μg/mL) staining for 20 min, sections were observed using a confocal laser scanning microscope (Leica TCS SPS, Germany) with a diode (50 mW) and DPSS (diode pumped solid state; 10 mW) laser light source.

### Distribution of PNTs In Vivo

To observe the uptake of PNTs at biomembranes and trace the distribution of delivered PNTs, thioflavin T (4 μM) pre-stained PNTs were administrated following the methods described in the oral and topically eye drop gene transfer in vivo section. Mice receiving no treatment served as control groups. Mice were sacrificed by cervical dislocation at 1 h after the first dose, and β-Gal or hRluc expressing tissues including the stomach, duodenum, liver, lung, brain, kidney, and cornea were removed and processed for cryosection following the methods described in the section of tissue section for pCMV-lacZ delivery and pCMV-hRluc delivery. After DAPI (1 μg/mL) staining for 20 min, sections were sealed with Leica CV Mount and observed by a fluorescence microscope (Olympus BX40, Japan) with a fixed exposure time.

### Southern Blot Analysis.

Stomach, duodenum, liver, and kidney were harvested at 1, 2, and 3 h after the oral first dose and at 4 h with the oral second dose at 3 h intervals of plasmid DNA or plasmid DNA formulated with PNTs. Total DNA was extracted from the homogenized tissues. Homogenate was lysed with 0.5% SDS and protease K (10 mg/mL) solution at 60° C. overnight. Total DNA was then phenol-chloroform extracted, ethanol precipitated at 4° C. overnight, washed with 70% ethanol, and dissolved with TE buffer. A 5 μg portion of total DNA from stomach and duodenum samples and a 50 μg portion of total DNA from liver and kidney samples were separated on 0.8% agarose gel by electrophoresis with a 1 kb ladder. The gels were then denatured with 0.5 N NaOH, followed by neutralized with 1 M Tris buffer (pH 7.4). DNA bands were then transferred to Nytran NY 13N membranes (Schleicher & Schuell, Dassel, Germany) and followed by a UV light cross-link at 254 nm with 0.15 J/cm² of energy. After pre-hybridization with ULTRAhyb hybridization buffer (Ambion, Austin, Tex.) for 4 h, membranes were incubated at 42° C. for 16 h with biotin-14-dATP (BioNick Labeling System, Invitrogen Life Technologies) labeled a CMV-lacZ DNA fragment which was obtained from the digestion of pCMV-lacZ with PstI restriction enzyme. Finally, membranes were performed with chemiluminescent detection using the Phototope-Star Detection Kit (New England BioLabs, Ipswich, Mass., USA) and then exposed to Kodak BioMax Light film (Kodak, Rochester, N.Y., USA).

Detection of lacZ and hRluc Genes mRNA

Forty-eight and seventy-two hours after the first oral dose of pCMV-lacZ/PNTs or pCMV-hRluc/PNT formulation, total RNA was extracted from the stomach, duodenum, liver, and kidney with TRIzol reagent (Invitrogen Life Technologies, Carlsbad, Calif., USA) according to the manufacturer's instructions. Total RNA (2.5 μg) was reverse-transcribed with SuperScript II reverse transcriptase (Invitrogen Life Technologies, Carlsbad, Calif., USA) primed with oligo-dT (10 μM). The amount of cDNA was quantified by RT-qPCR the same as described in the loading efficiency of pCMV-lacZ/PNTs section. For hRluc mRNA analysis, the primers for Renilla luciferase: forward: 5′-TCC CTG ATC TGA TCG GAATGG G-3′ (SEQ ID NO: 3), and reverse: 5′-CTT GGT GCT CGT AGG AGTAGT G-3′ (SEQ ID NO: 4), were used.

Ex Vivo Bioluminescence Imaging of hRluc

To image the pCMV-hRluc delivery, mice were anesthetized with a mixture of oxygen/isofluorane and received with 0.7 mg/kg of colenterazine (Biotium Inc., Hayward, Calif., USA) by cardiac puncture. The photon emission transmitted from dissected organs was measured with an IVIS Imaging System 200 Series (Xenogen, Alameda, Calif.) with a fixed exposure time. The intensity was recorded as a maximum (photons/s/cm²/sr).

### Results

Characterization of pCMV-lacZ/PNTs.

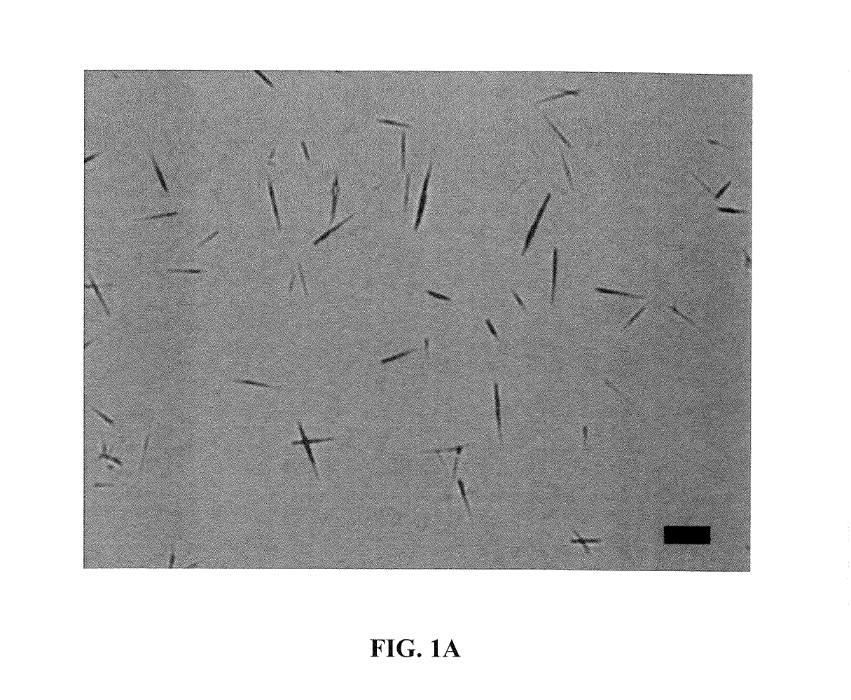

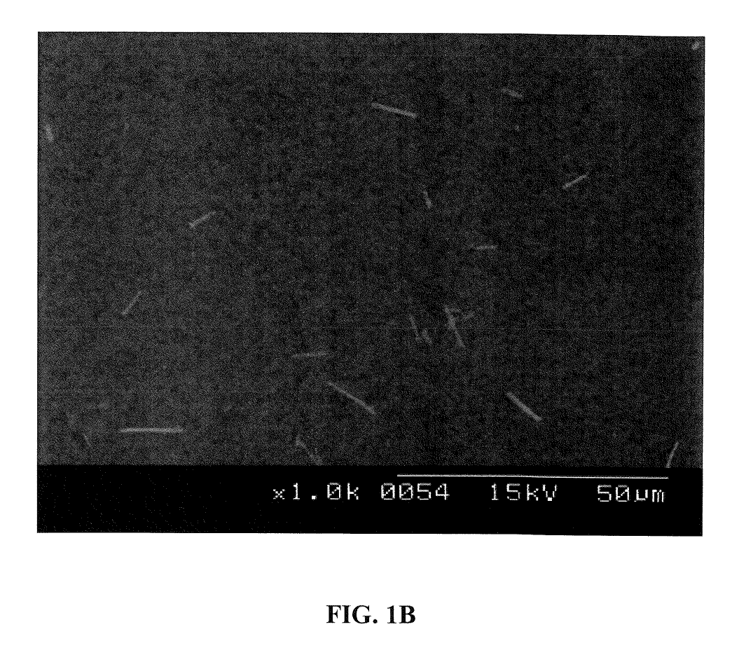

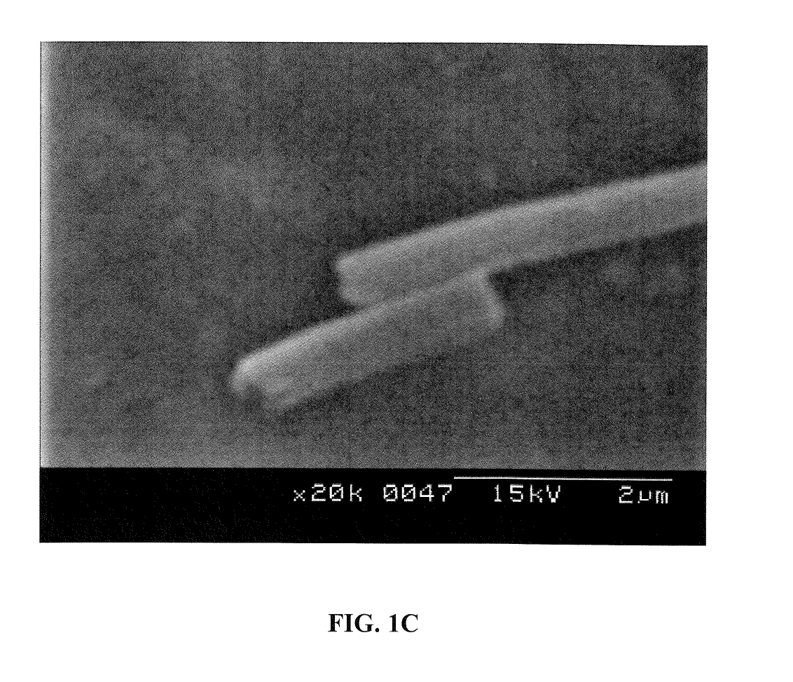

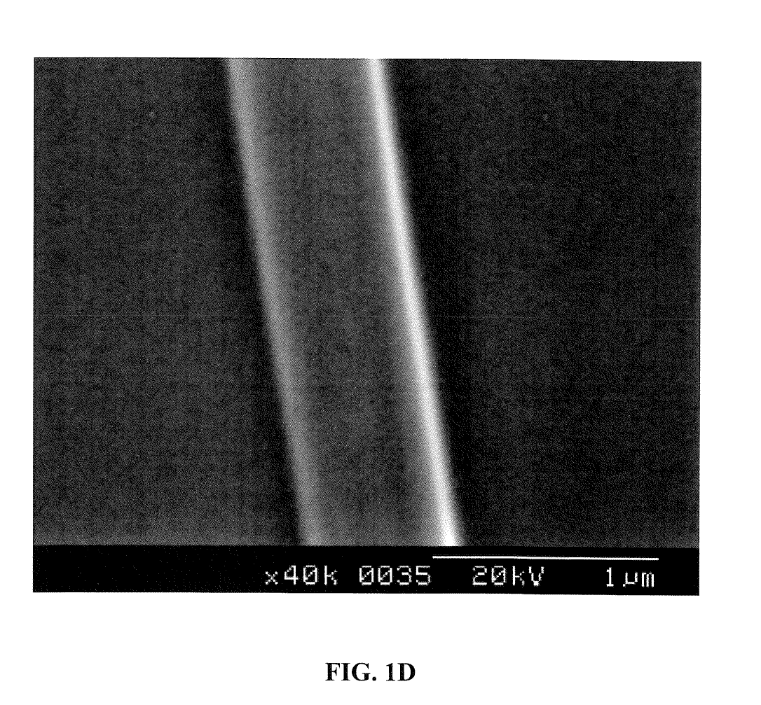

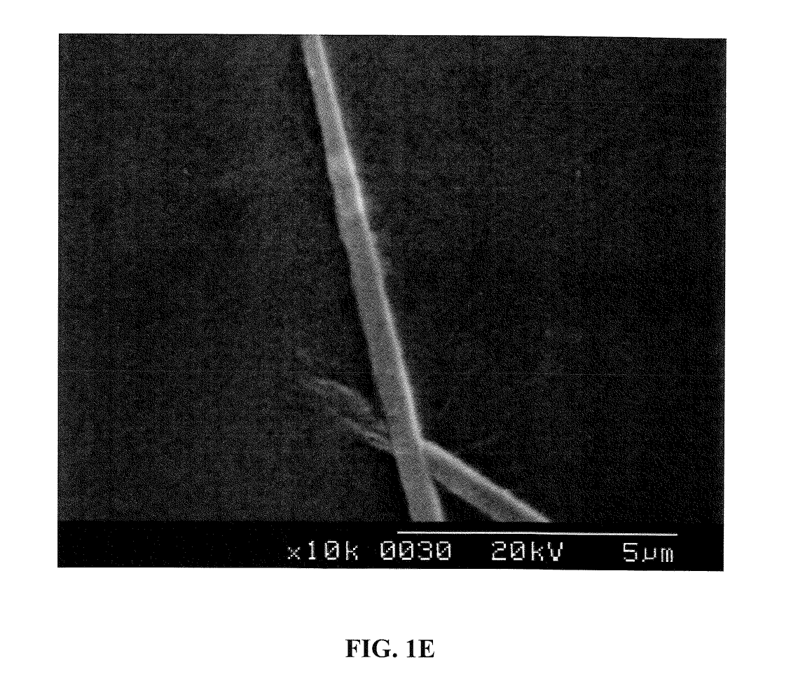

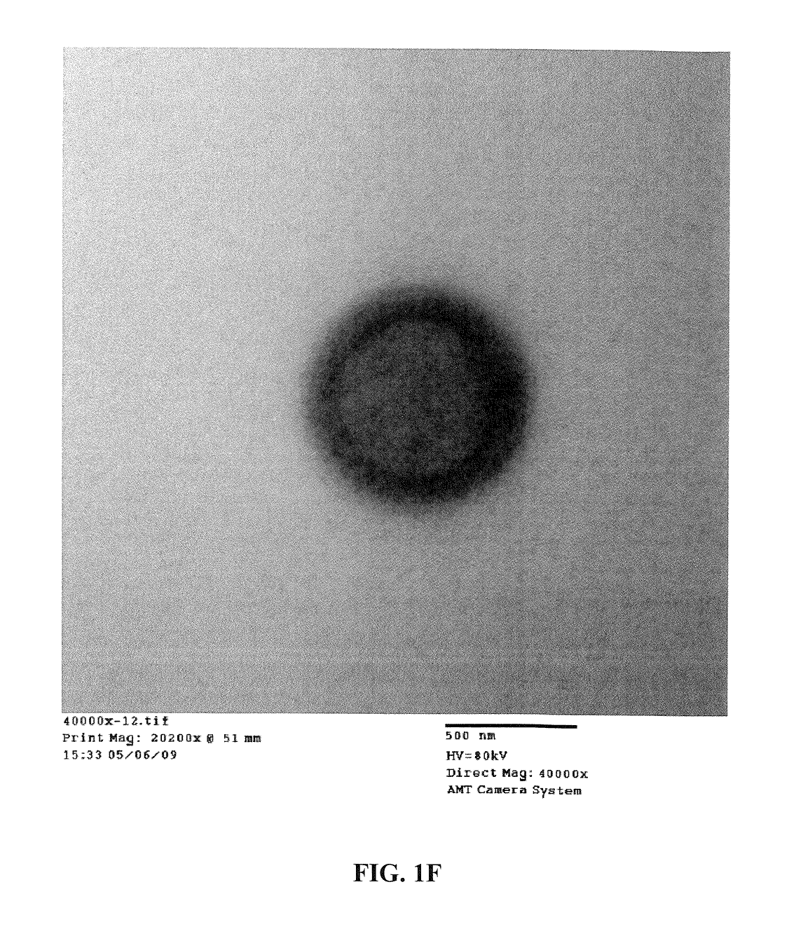

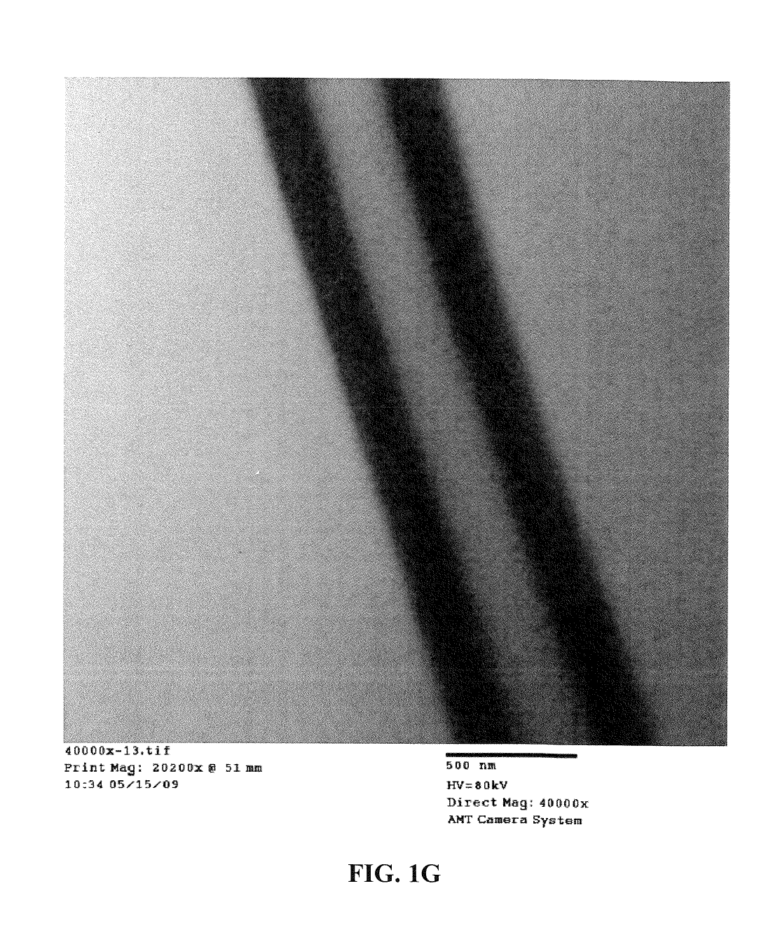

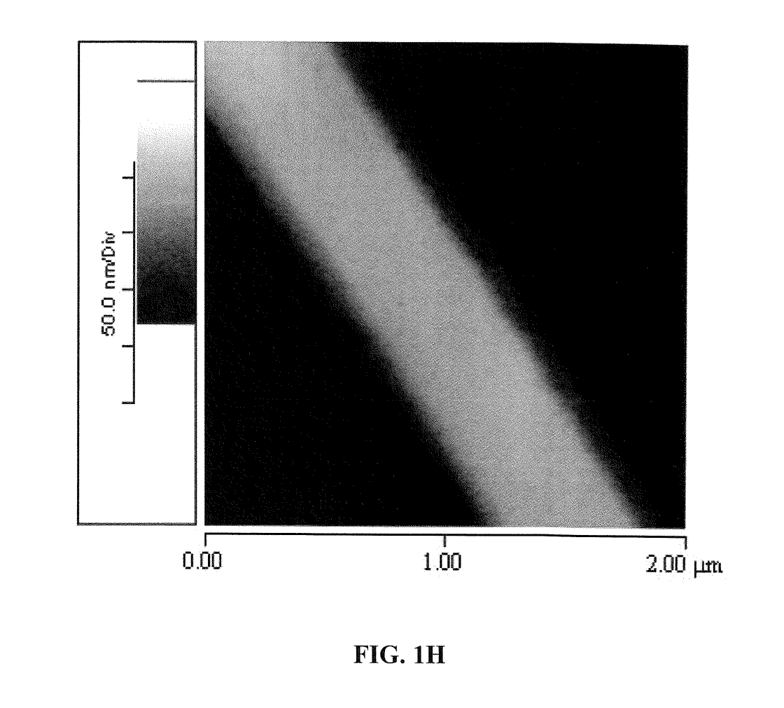

With reference to part A of FIG. 1 for an optical microscope view, parts B-E of FIG. 1 for scanning electron microscope (SEM) views, parts F-G of FIG. 1 for transmission electron microscope (TEM) views, and parts H-I of FIG. 1 for atomic force microscope (AFM) views and cross section of AFM image along the line in part H of FIG. 1, of peptide nanotube devices prepared with trifluoroacetic acid (TFA) in accordance with an embodiment of the present invention, the needle-shaped PNTs, prepared with TFA, composed of cyclo-(D-Trp-Tyr) peptide were observed by optical microscopy in part A of FIG. 1. The PNTs prepared with TFA were appeared to be 500 nm in width and 15 μm in length. In a preferred embodiment, the PNTs prepared with TFA may be 100-800 nm in width and 1-20 μm in length. Referring to parts B-G of FIG. 1, higher magnification images of the scanning electron microscope (SEM) and transmission electron microscope (TEM) showed that these needle-shaped PNTs prepared with TFA had a hollow tubular structure with an open circle end. The SEM imaging in part E of FIG. 1 revealed some small nanotubes with estimated 20-30 nm diameters around the bundle of multi-walled PNTs prepared with TFA, indicating the obtained PNTs prepared with TFA may be formed by single nanotubes bundled or aggregated together. Images of AFM in parts H-I of FIG. 1 further showed that cyclo-(D-Trp-Tyr) peptide PNTs prepared with TFA were long tubes with approximately 700 nm in width and 180 nm in height.

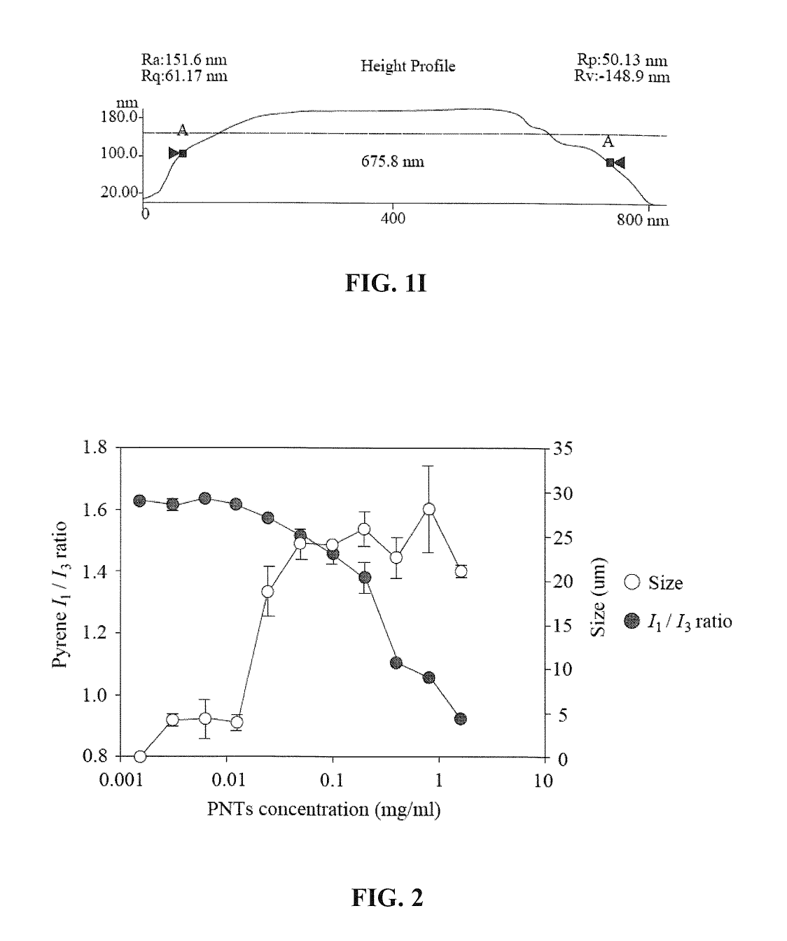

With reference to FIG. 2 for a schematic view of self-association and size distribution of peptide nanotube devices prepared with TFA in accordance with an embodiment of the present invention, the self-association of PNTs prepared with TFA was evaluated using pyrene as a fluorescence probe. The critical association concentration (CAC) was determined by the pyrene I₁/I₃ ratio, a well-known property reflecting the microenvironment polarity. Referring to the solid circular symbol, results showed that the CAC of PNTs prepared with TFA was above the 0.01 mg/mL concentration. To further evaluate the formation of PNTs prepared with TFA, the sizes of PNTs prepared with TFA at concentrations of 1.6 μg/mL to 1.6 mg/mL were analyzed by quasielastic laser dynamic light scattering (DLS). Referring to the open circular symbol, the results showed that the overall sizes of PNTs prepared with TFA were between 20 and 30 μm when concentrations of PNTs prepared with TFA were above the CAC, while the overall sizes of PNTs prepared with TFA decreased dramatically to below 5 μm as the concentrations of PNTs prepared with TFA were below the CAC. These results indicated that the assembly of PNTs prepared with TFA depended on the peptide concentration. In addition, referring Table 1, the overall size of PNTs prepared with TFA at a 1.5 mg/mL concentration was averaged at 17 μm measured by DLS; this was similar to the length estimated on the images obtained by optical and SEM microscopes. To ensure that PNTs prepared with TFA were remained in a tubular shape, PNTs prepared with TFA at this concentration (1.5 mg/mL) were used for all further studies including the in vitro duodenal permeability and in vivo oral delivery.

TABLE 1
PNTs prepared with TFA

| | | microscope | | |
|---|---|---|---|---|
| | | Width | | Zeta potential |
| Formulation | DLS size (nm) | (nm) | Length (nm) | (mV) |
| PNTs | 17408.1 ± 2242.8 | 100-800 | 1000-20000 | −7.3 ± 4.3 |
| pCMV-lacZ | 56.0 ± 7.4 | N.A. | N.A. | −50.2 ± 15.1 |
| pCMV- | 19249.1 ± 3706.0 | 100-800 | 1000-20000 | −56.5 ± 18.0 |
| lacZ/PNTs | | | | |

N.A.: not available

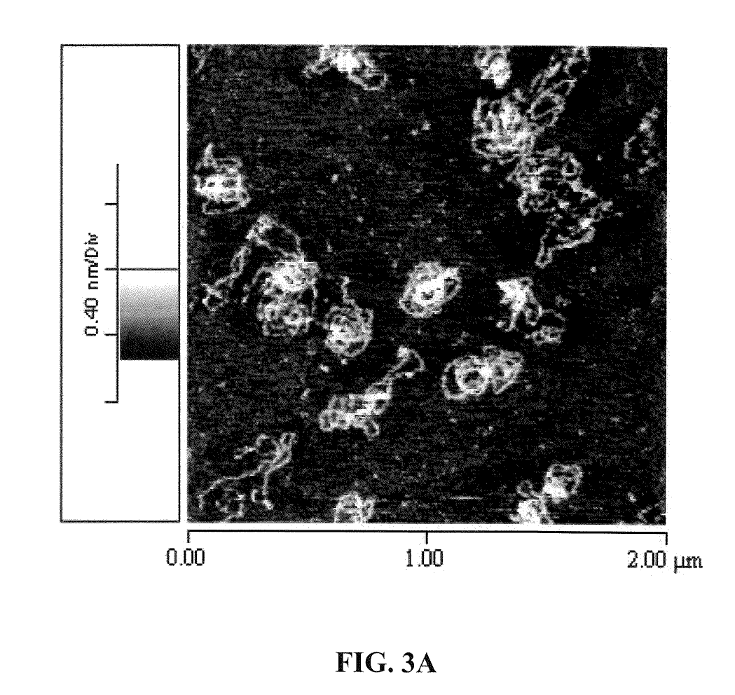

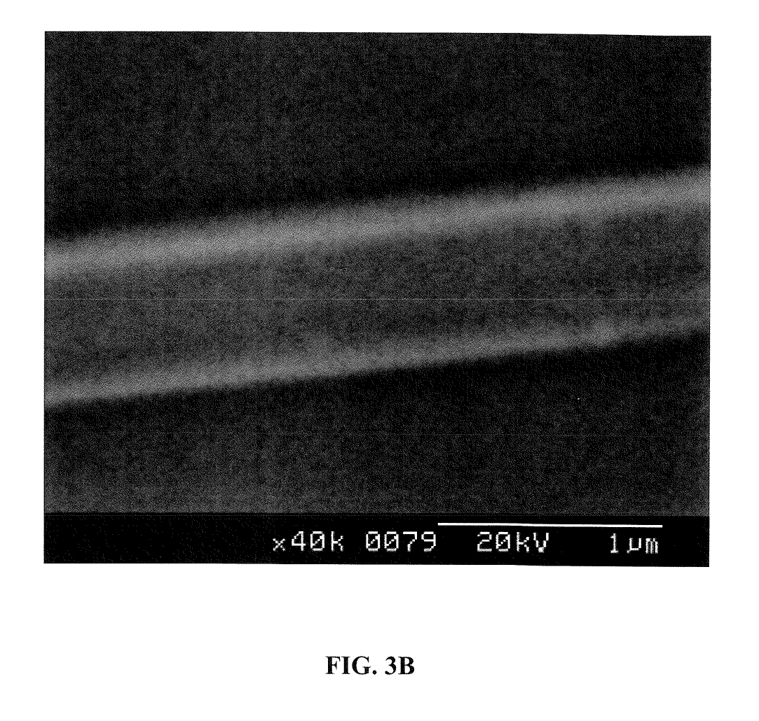

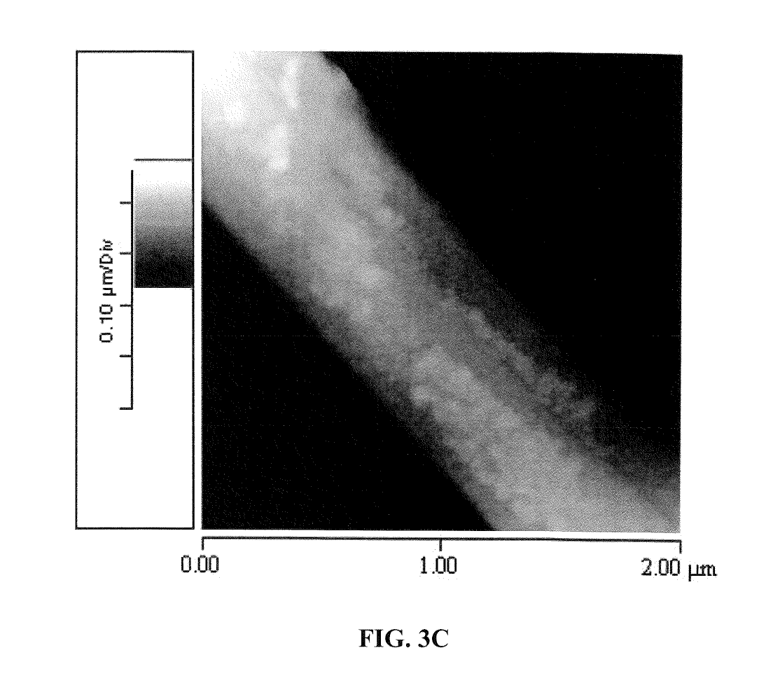

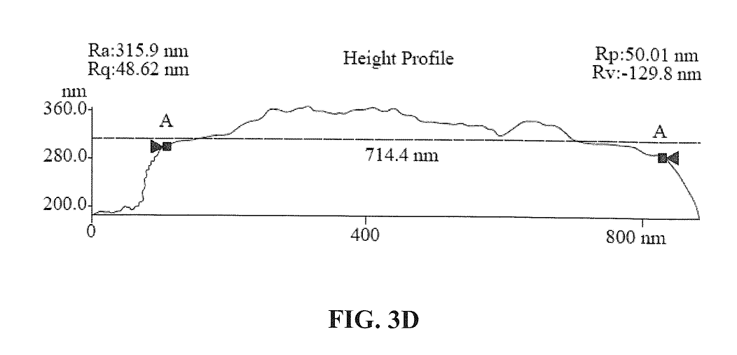

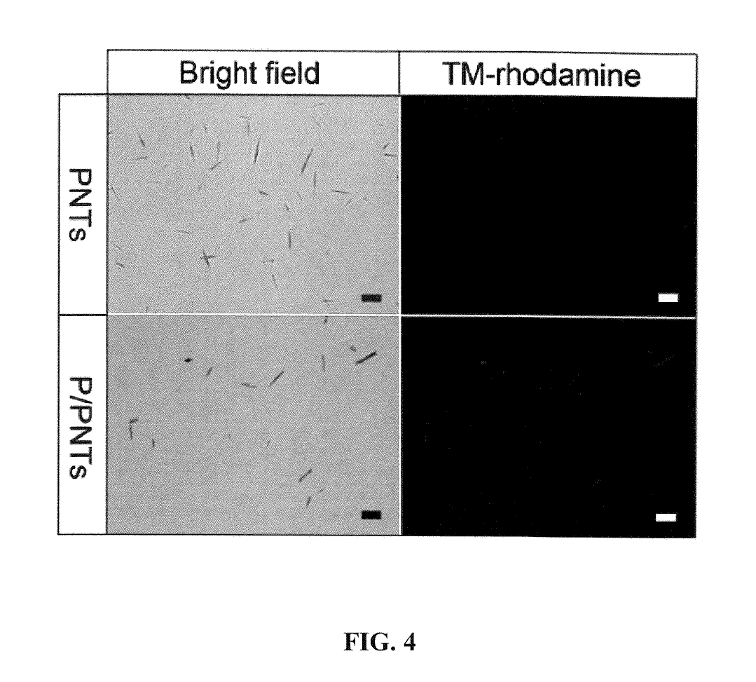

Furthermore, as shown in Table 1, the overall size of pCMV-lacZ/PNT formulation was averaged to be 19 μm measured by DLS and similar to the length of pCMV-lacZ/PNT formulation observed by optical and SEM microscopes. The similar size distribution of PNTs prepared with TFA and pCMV-lacZ/PNTs prepared with TFA suggested that the presence of plasmid DNA did not affect the sizes of PNTs prepared with TFA. To analyze the effect on surface charge, the zeta potential of the pCMV-lacZ/PNT formulation was measured. The results revealed that the zeta potential of pCMV-lacZ or PNTs prepared with TFA alone in water was −50.2 mV and −7.3 mV, respectively. The zeta potential was shifted to −56.5 mV when pCMV-lacZ formulated with PNTs prepared with TFA. The mono-dispersion and more negative zeta potential of pCMV-lacZ/PNTs prepared with TFA indicated that the plasmid DNA was associated on the surface of PNTs prepared with TFA. With reference to parts A-D of FIG. 3 for an AFM view of pCMV-lacZ plasmid alone, a SEM view of pCMV-lacZ plasmid formulated with peptide nanotube prepared with TFA, an AFM views of pCMV-lacZ plasmid formulated with peptide nanotube devices prepared with TFA, and a cross section of AFM image along the line in part C of FIG. 3, respectively, in accordance with an embodiment of the present invention, to further confirm the association of DNA on PNT surface, SEM and AFM imagings of pCMV-lacZ/PNTs prepared with TFA were performed. Referring to parts B and C of FIG. 3, results showed that aggregated particles were found on the surface of PNTs prepared with TFA. In addition, referring to part D of FIG. 3, the rugged cross section of pCMV-lacZ/PNT formulation was imaged by an AFM in contrast to the smooth surface of PNTs prepared with TFA alone shown in parts D, H and I of FIG. 1. Furthermore, with reference to FIG. 4 for a fluorescence microscope view of TM-rhodamine labeled pCMV-lacZ plasmid formulated with peptide nanotube devices prepared with TFA, TM-rhodamine labeled pCMV-lacZ (P/PNTs) was also associated with PNTs prepared with TFA detected by a fluorescence microscope.

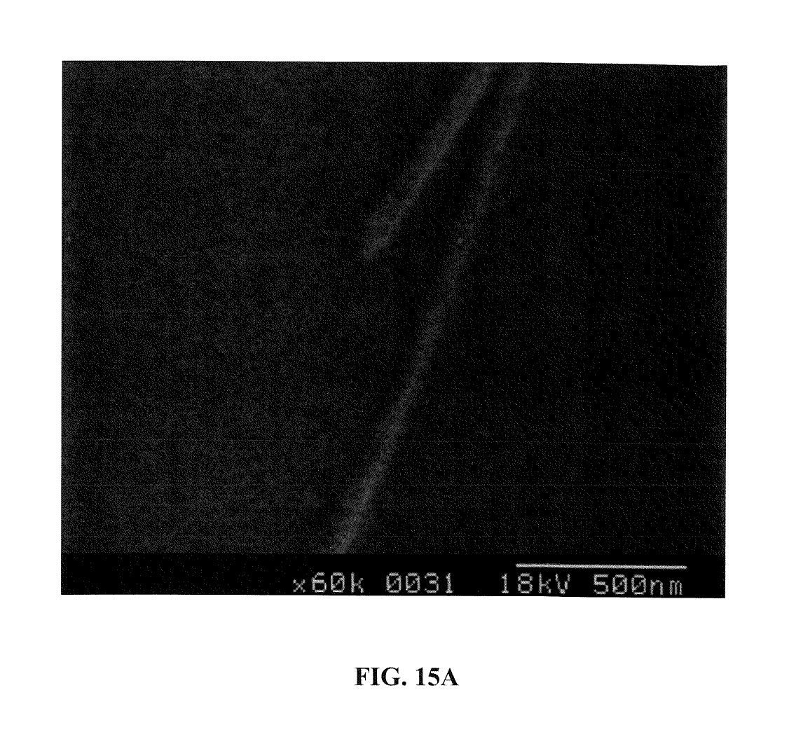

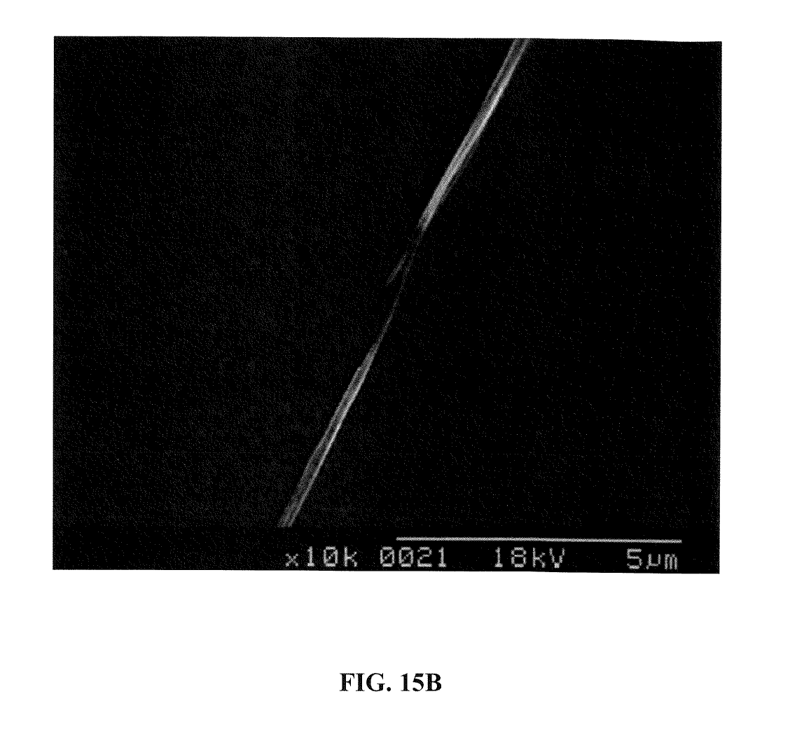

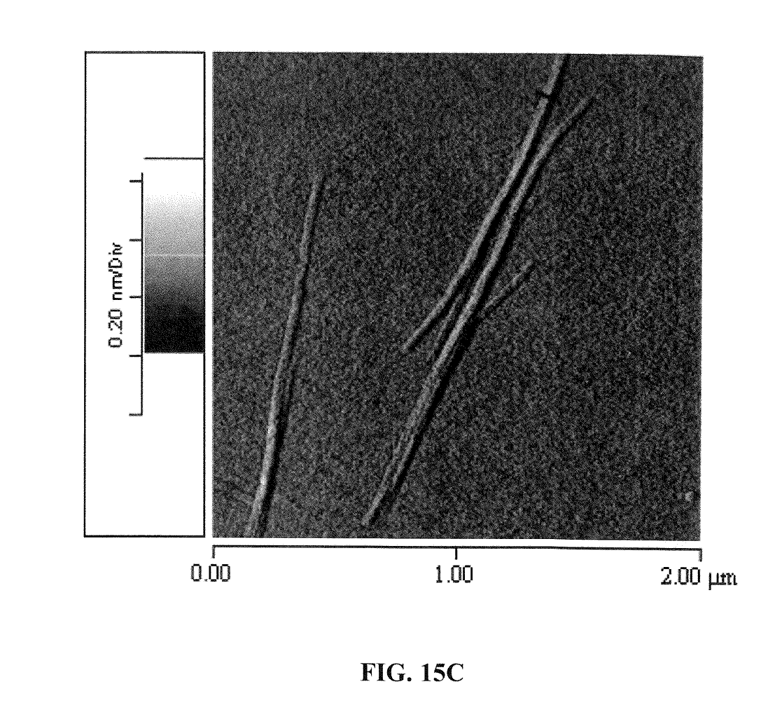

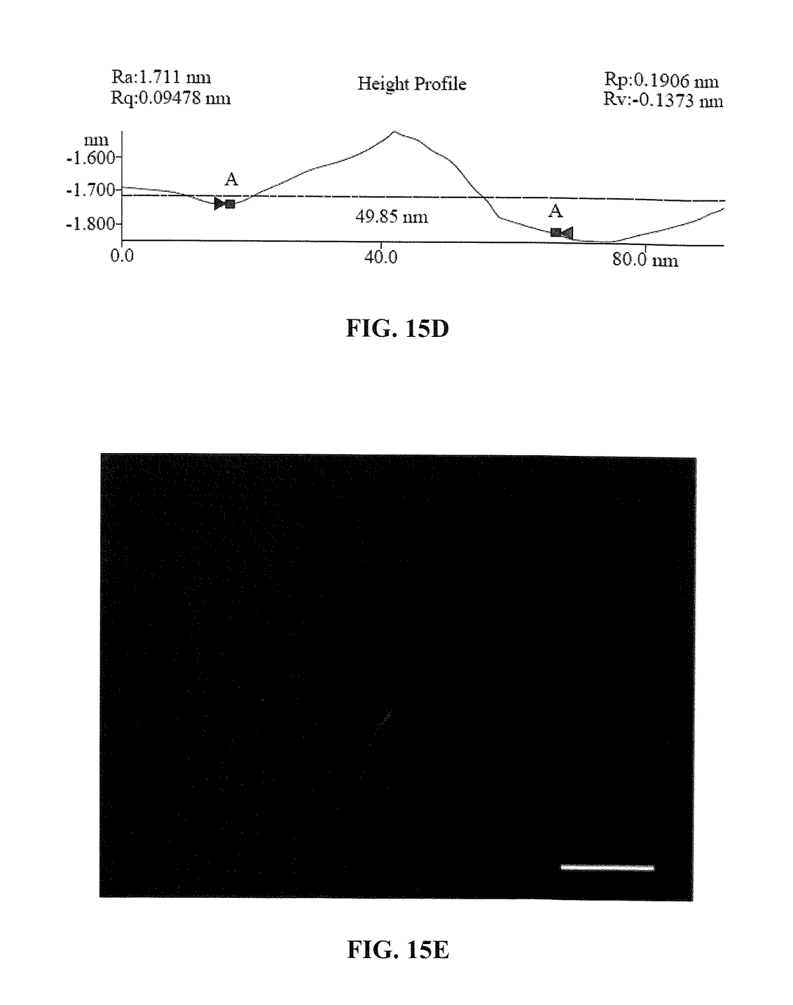

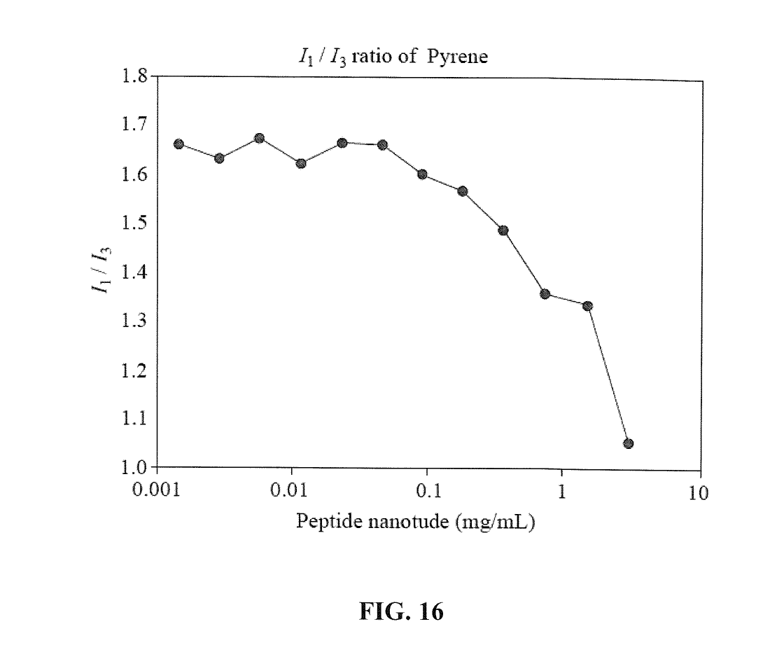

In addition, with reference to parts A and B of FIG. 15 for scanning electron microscope (SEM) views, parts C and D of FIG. 15 for an atomic force microscope (AFM) view and a cross section of AFM image along the line in part C of FIG. 15, and part E of FIG. 15 for an fluorescence microscope view of peptide nanotube devices prepared with ethanol in accordance with an embodiment of the present invention, the needle-shaped PNTs composed of cyclo-(D-Trp-Tyr) appeared to be 50-200 nm in width and 400-2000 nm in length, observed by SEM and AFM. It showed that these needle-shaped PNTs revealed some small peptide nanotubes with estimated 20-30 nm diameters around the bundle of multi-walled PNTs, indicating the obtained PNTs may be formed by single nanotubes bundled or aggregated together.

TABLE 2
PNTs prepared with ethanol

| | | Microscope | | |
|---|---|---|---|---|
| | | Width | Length | Zeta potential |
| Formulation | DLS Size (nm) | (nm) | (nm) | (mV) |
| PNTs | 2276 ± 507.49 | 30-200 | 1000-3000 | 4.08 ± 1.23 |
| pCMV-hRluc | 68.9 ± 3.29 | N.A | N.A | −35.66 ± 2.59 |
| pCMV- | 1726.67 ± 603.63 | 30-200 | 1000-3000 | −43.52 ± 14.62 |
| hRluc/PNTs | | | | |

N.A.: not available

The self-association of PNTs was evaluated using pyrene as fluorescence probe. The critical association concentration (CAC) was determined by the pyrene I₁/I₃ ratio, a well-known property reflecting the microenvironment polarity. Referring to Table 2, results showed that the CAC of PNTs was above 0.01 mg/ml concentration. To further evaluate the formation of PNTs, the sizes of PNTs at concentrations of 1.5 mg/ml were analyzed by quasielastic laser dynamic light scattering (DLS). The results showed that the overall sizes of PNTs were between 1-3 μm. The similar size distribution of PNTs and pCMV-hRluc/PNTs suggested that the presence of plasmid DNA did not affect the sizes of PNTs. To analyze the effect on surface charge, the Zeta potential of the pCMV-hRluc/PNTs formulation was measured. Table 2 revealed that the Zeta potential of pCMV-hRluc or PNTs alone in water was −35 mV and 4 mV, respectively. The Zeta potential was shift to −43 mV when pCMV-hRluc formulated with PNTs. The mono-dispersion and negative Zeta potential of pCMV-hRluc/PNTs indicated that the plasmid DNA was associated on the surface of PNTs.

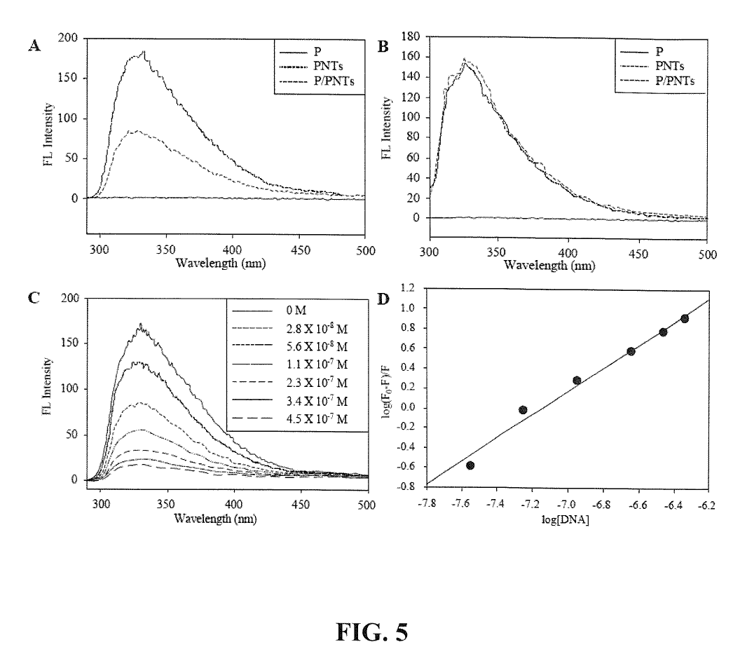

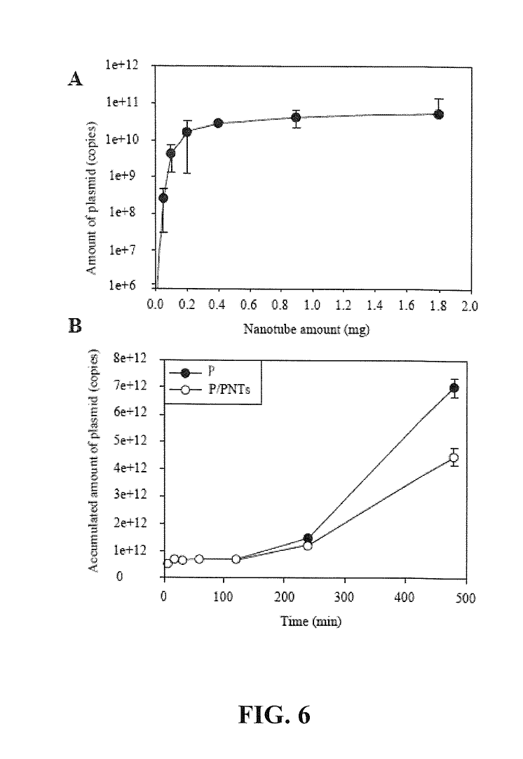

To understand the involvement of Trp and Tyr residues of PNTs prepared with TFA in association with DNA, the fluorescence emission spectra of PNTs with or without DNA was examined. With reference to parts A-C of FIG. 5 for emission fluorescence spectra of pCMV-lacZ plasmid formulated with peptide nanotube devices prepared with TFA in accordance with an embodiment of the present invention, the emission intensity contributed by both Trp and Tyr of PNTs prepared with TFA, with an excitation at 280 nm, was significantly decreased when DNA added, as shown in part A of FIG. 5. However, referring to part B of FIG. 5, the emission intensity of fluorescence with excitation at 295 nm which was specific for Trp in PNTs prepared with TFA was not influenced by the addition of DNA. The results indicated that quenching of the emission spectra with excitation at 280 nm was due to DNA interaction with Tyr but not Trp residues in PNTs prepared with TFA. Furthermore, referring to part C of FIG. 5, the level of quenching at Tyr fluorescence emission spectra was found augmented with increasing concentration of DNA used. With reference to part D of FIG. 5 for a linear plot of pCMV-lacZ plasmid formulated with peptide nanotube devices prepared with TFA in accordance with an embodiment of the present invention, the binding constant (K) of Tyr residues in PNT to DNA and the mole fraction of bound DNA were calculated to be 3.2×10⁸ M⁻¹ and 1.2 mole fraction of DNA bound to Tyr, respectively. Furthermore, with reference to part A of FIG. 6 for a schematic view of qPCR quantification analysis of the amount of pCMV-lacZ absorbed in peptide nanotube devices prepared with TFA in accordance with an embodiment of the present invention, the amount of plasmid DNA in the PNTs-formulated complexes quantified by qPCR was 3×10¹⁰ copies DNA/mg PNTs.

With reference to part B of FIG. 6 for a schematic view of pCMV-lacZ release profile in peptide nanotube devices prepared with TFA in accordance with an embodiment of the present invention, the release rate of DNA with PNT formulation was evaluated by using a Franz diffusion cell with a 0.2 μm pore size of the membrane. The accumulated amount of released DNA from PNT formulation versus time in minutes was shown in part B of FIG. 6. The rate of DNA released was calculated by the least-squares Higuchi method, Mᵣ/M_{∞}=kt^{1/2}, and to be 3.57×10¹¹ copies DNA/t^{1/2}. However, the release rate of DNA without PNT formulation was 5.92×10¹¹ copies DNA/t^{1/2}. These results indicated that DNA formulated with cyclo-(D-Trp-Tyr) peptide PNTs possesses a slow release property.

Stability of pCMV-lacZ/PNTs Prepared with TFA with DNase I, Simulated Gastric Acid, or Bile Digestion

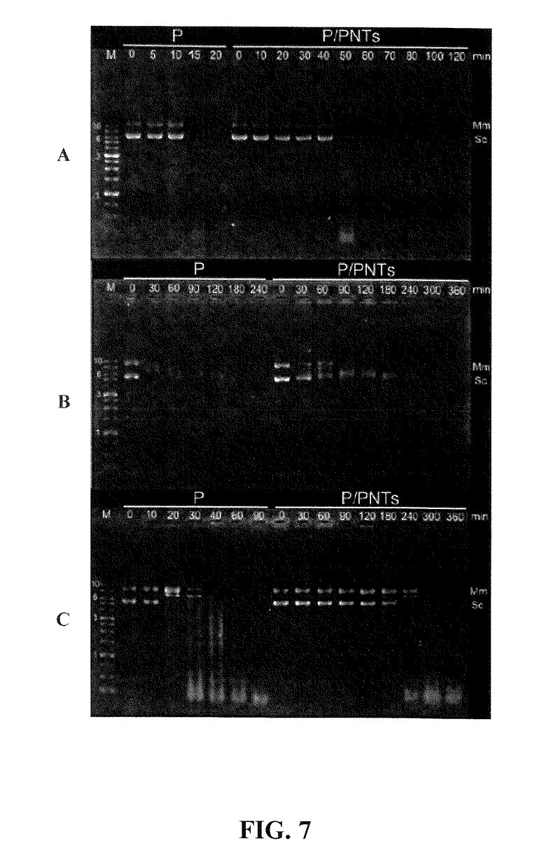

To determine whether the pCMV-lacZ/PNT formulation would enhance the stability of DNA against enzymatic, acid, and bile degradations, an in vitro DNase I, simulated gastric acid, and bile digestion assay was carried out by the incubation of DNase I, simulated gastric acid, or bile with PNTs-formulated DNA at 37° C. With reference to parts A-C of FIG. 7 for schematic views of the stability of pCMV-lacZ formulated with peptide nanotube devices prepared with TFA with treatment of DNase I, gastric acid and bile, respectively, in accordance with an embodiment of the present invention, the supercoiled pCMV-lacZ with a size of 7.2 kb was observed from DNase I digestion, simulated gastric acid hydrolysis, and bile digestion for 50, 60, and 180 min with PNTs, respectively. However, naked DNA was completely digested soon after incubation with DNase I within 10 min, with simulated gastric acid within 30 min, and with bile within 60 min.

Stability of PNTs with Simulated Gastric Acid Treatment

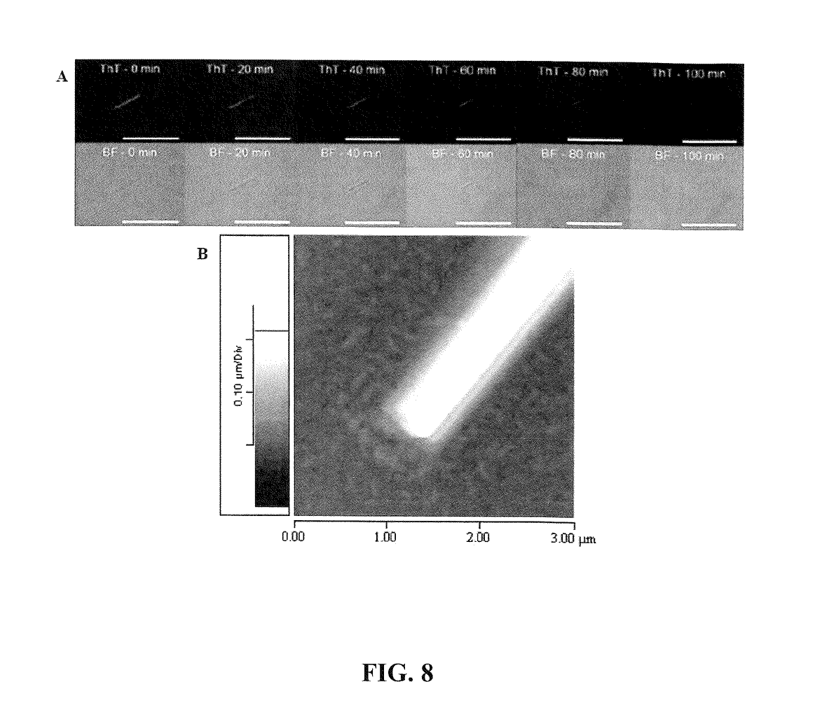

To evaluate the stability of PNTs prepared with TFA after oral delivery, the inventors incubated thioflavin T pre-stained PNTs prepared with TFA with simulated gastric acid to mimic the in vivo situation. Parts A and B of FIG. 8 are fluorescence microscope and bright field views of the stability of peptide nanotube devices prepared with TFA with treatment of gastric acid for indicated time intervals, and an AFM view of the stability of peptide nanotube devices prepared with TFA with treatment of gastric acid, respectively. Referring to part A of FIG. 8, the results showed that a decrease in both length and width of PNTs prepared with TFA was detected over the tested period of time in the presence of simulated gastric acid. Referring to part B of FIG. 8, the result of AFM imaging also observed the degradation of PNTs prepared with TFA when treated with gastric acid, indicating the occurrence of degradation.

### In Vitro Duodenal Penetration Studies

To evaluate whether the concentration of PNTs prepared with TFA affected the permeability of DNA in small intestine after oral administration, in vitro duodenal penetration was performed with a Franz cell. As shown in Table 3, the apparent permeability coefficient of plasmid DNA was significantly increased from 49.2±21.6×10⁻¹⁰ cm/s for naked DNA to 395.6±142.2×10⁻¹⁰ cm/s for DNA formulated with 1.5 mg/mL of PNTs penetrating from apical to basolateral direction at 37° C. The apparent permeability coefficients of plasmid formulated with PNT at 4° C. or in the presence of sodium azide were also analyzed to investigate the energy effect. Additionally, penetrating from basolateral to apical, the reverse direction was performed. The results showed that the apparent permeability coefficient of PNT formulated plasmid DNA was decreased at 4° C. or in the presence of sodium azide compared to that performed at 37° C., indicating the energy-dependent penetration. The apparent permeability coefficient was also decreased when penetration processed in the reverse direction.

TABLE 3
PNTs prepared with TFA

| | PNT | |
|---|---|---|
| | concentration | Apparent permeability |
| Formulation | (mg/mL) | coefficient × 10⁻¹⁰ (cm/s) |
| pCMV-lacZ | 0 | 49.2 ± 21.6 (n = 15)^{†} |
| pCMV-lacZ/PNTs (37° C.) | 0.01 | 53.7 ± 48.3 (n = 4)^{†} |
| | 0.2 | 158.2 ± 94.2 (n = 4)^{†} |
| | 0.8 | 403.1 ± 235.7 (n = 4) |
| | 1.5 | 395.6 ± 142.2 (n = 18) |
| pCMV-lacZ/PNTs (4° C.) | 1.5 | 8.1 ± 1.7 (n = 5)^{†} |
| pCMV-lacZ/PNTs (NaN₃) | 1.5 | 81.6 ± 23.2 (n = 6)^{†} |
| pCMV-lacZ/PNTs (reverse) | 1.5 | 34.2 ± 43.2 (n = 6)^{†} |

^{†}significant difference (p < 0.05) compared with the apparent permeability coefficient of plasmid DNA formulated with 1.5 mg/mL of PNTs at 37° C.

### Oral Gene Transfer In Vivo

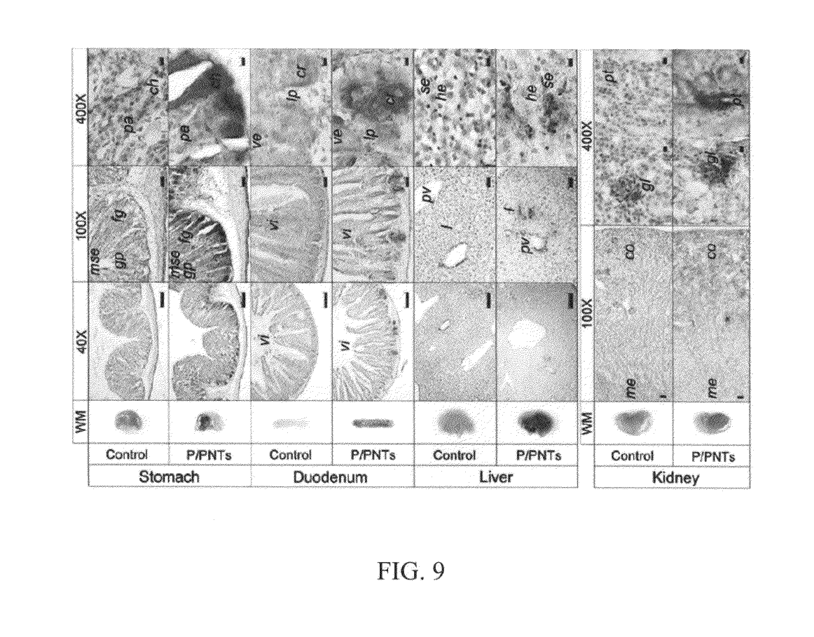

After 48 and 72 h of the first oral pCMV-lacZ/PNTs dose, the mice were sacrificed, and the β-Gal activity in various organs, including the duodenum, testis, kidney, stomach, heart, liver, brain, lung, spinal cord, and spleen, were evaluated using CPRG as the substrate. As shown in Table 4, results showed that the β-Gal activity significantly increased in the kidney (41%) at 48 h and in the stomach (49%), duodenum (63%), and liver (46%) at 72 h after oral administration of the first dose of pCMV-lacZ/PNTs (p<0.05). No β-Gal activity was detected in all tissues after oral administration of plasmid DNA or PNTs prepared with TFA alone compared with that in the control group. With reference to FIG. 9 for histological views of X-Gal staining of the various tissues of nude mice with oral delivery of pCMV-lacZ formulated with peptide nanotube devices prepared with TFA in accordance with an embodiment of the present invention, results of histological analysis showed that β-Gal activity, which was indicated as blue-green color, was found in the stomach, duodenum, liver, and kidney. Wherein, mse denoted mucosa surface epithelium, gp denoted gastric pits, fg denoted fundus gland, pa denoted parietal cells, ch denoted chief cells, ye denoted villous epithelium, lp denoted lamina propria, cr denoted crypt cells, vi denoted duodenal villi, l denoted lobules, he denoted hepatocyte, se denoted sinusoidal endothelial cells, pv denoted portal vein, gl denoted glomerular, and pt denoted proximal tubular. There were no pathological and inflammatory characteristics observed in all images of tissue sections from animals receiving pCMV-lacZ/PNT formulation.

TABLE 4
PNTs prepared with TFA

| | Time | β-Gal activity (mU/mg protein) in tissues | | | | |
|---|---|---|---|---|---|---|
| Formulation | (h) | duodenum | testis | kidney | stomach | heart |
| pure control | 48 | 0.49 ± 0.07 | 0.66 ± 0.09 | 1.06 ± 0.18 | 0.51 ± 0.15 | 0.23 ± 0.09 |
| (n = 7) | | | | | | |
| pCMV-lacZ | | 0.56 ± 0.13 | 0.83 ± 0.14 | 0.93 ± 0.27 | 0.51 ± 0.10 | 0.12 ± 0.03 |
| alone | | | | | | |
| (n = 5) | | | | | | |
| PNTs alone | | 0.55 ± 0.12 | 0.80 ± 0.22 | 1.17 ± 0.14 | 0.49 ± 0.21 | 0.29 ± 0.20 |
| (n = 8) | | | | | | |
| pCMV- | | 0.65 ± 0.44 | 0.79 ± 0.42 | 1.49 ± 0.34^{†} | 0.43 ± 0.16 | 0.25 ± 0.04 |
| lacZ/PNTs | | | | | | |
| (n = 7) | | | | | | |
| pure control | 72 | 0.49 ± 0.12 | 0.66 ± 0.18 | 1.06 ± 0.13 | 0.51 ± 0.08 | 0.23 ± 0.03 |
| (n = 6) | | | | | | |
| pCMV-lacZ | | 0.50 ± 0.17 | 0.58 ± 0.13 | 1.14 ± 0.14 | 0.44 ± 0.08 | 0.23 ± 0.04 |
| alone | | | | | | |
| (n = 8) | | | | | | |
| PNTs alone | | 0.54 ± 0.19 | 0.59 ± 0.07 | 1.16 ± 0.11 | 0.51 ± 0.07 | 0.23 ± 0.07 |
| (n = 6) | | | | | | |
| pCMV- | | 0.80 ± 0.17^{†} | 0.64 ± 0.15 | 1.31 ± 0.17 | 0.76 ± 0.27^{†} | 0.22 ± 0.04 |
| lacZ/PNTs | | | | | | |
| (n = 6) | | | | | | |
| | | β-Gal activity (mU/mg protein) in tissues | | | | |
| | Time | | | | spinal | |
| Formulation | (h) | liver | brain | lung | cord | spleen |
| pure control | 48 | 0.35 ± 0.08 | 0.28 ± 0.06 | 0.64 ± 0.20 | 0.40 ± 0.16 | 1.02 ± 0.32 |
| (n = 7) | | | | | | |
| pCMV-lacZ | | 0.29 ± 0.06 | 0.21 ± 0.03 | 0.53 ± 0.08 | 0.25 ± 0.10 | 1.03 ± 0.43 |
| alone | | | | | | |
| (n = 5) | | | | | | |
| PNTs alone | | 0.40 ± 0.13 | 0.42 ± 0.30 | 0.82 ± 0.34 | 0.72 ± 0.72 | 1.29 ± 0.37 |
| (n = 8) | | | | | | |
| pCMV- | | 0.32 ± 0.12 | 0.24 ± 0.03 | 0.59 ± 0.09 | 0.34 ± 0.02 | 1.01 ± 0.22 |
| lacZ/PNTs | | | | | | |
| (n = 7) | | | | | | |
| pure control | 72 | 0.35 ± 0.05 | 0.28 ± 0.04 | 0.64 ± 0.04 | 0.40 ± 0.04 | 1.02 ± 0.08 |
| (n = 6) | | | | | | |
| pCMV-lacZ | | 0.36 ± 0.07 | 0.27 ± 0.03 | 0.68 ± 0.07 | 0.44 ± 0.08 | 1.07 ± 0.26 |
| alone | | | | | | |
| (n = 8) | | | | | | |
| PNTs alone | | 0.36 ± 0.07 | 0.26 ± 0.02 | 0.85 ± 0.35 | 0.41 ± 0.04 | 0.89 ± 0.16 |
| (n = 6) | | | | | | |
| pCMV- | | 0.51 ± 0.09^{†} | 0.28 ± 0.02 | 0.80 ± 0.10 | 0.39 ± 0.04 | 1.15 ± 0.15 |
| lacZ/PNTs | | | | | | |
| (n = 6) | | | | | | |

^{†}significant difference (p < 0.05) compared with the same tissue of the control groups

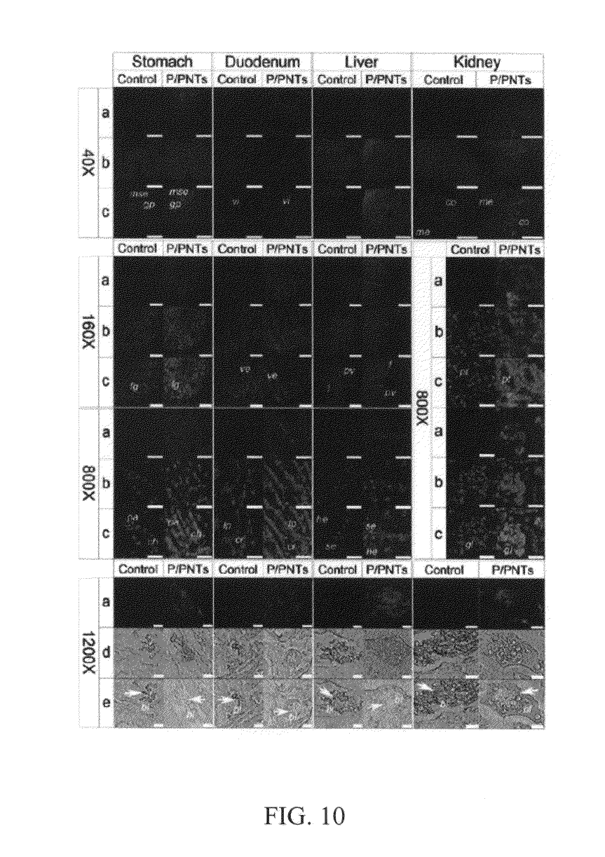

To trace the presence of plasmid DNA in stomach, duodenum, liver, and kidney, mice was orally delivered with TM-rhodamine-labeled pCMV-lacZ formulated with PNTs prepared with TFA. After 1 h of the first dose, mice were sacrificed, and the organs were processed for paraffin sectioning and for confocal laser scanning microscope imaging. With reference to FIG. 10 for fluorescence microscope views of the various tissues of nude mice with oral delivery of TM-rhodamine labeled pCMV-lacZ formulated with peptide nanotube devices prepared with TFA in accordance with an embodiment of the present invention, TM-rhodamine signals were found in stomach, duodenum, liver, and kidney where β-Gal enzymatic activity was also detected. Section a is TM-Rhodamine images (red), section b is DAPI images (blue), section c is merged images of images in sections a and b, section d is bright field images merged with the DAPI image, and section e is TM-rhodamine image merged with the image in section d. Wherein, mse denoted mucosa surface epithelium, gp denoted gastric pits, fg denoted fundus gland, pa denoted parietal cells, ch denoted chief cells, ye denoted villous epithelium, lp denoted lamina propria, cr denoted crypt cells, vi denoted duodenal villi, l denoted lobules, he denoted hepatocyte, se denoted sinusoidal endothelial cells, pv denoted portal vein, gl denoted glomerular, pt denoted proximal tubular, and bl with arrows denoted blood. In addition, TM-rhodamine was found in blood circulating in the stomach, duodenum, liver, and kidney.

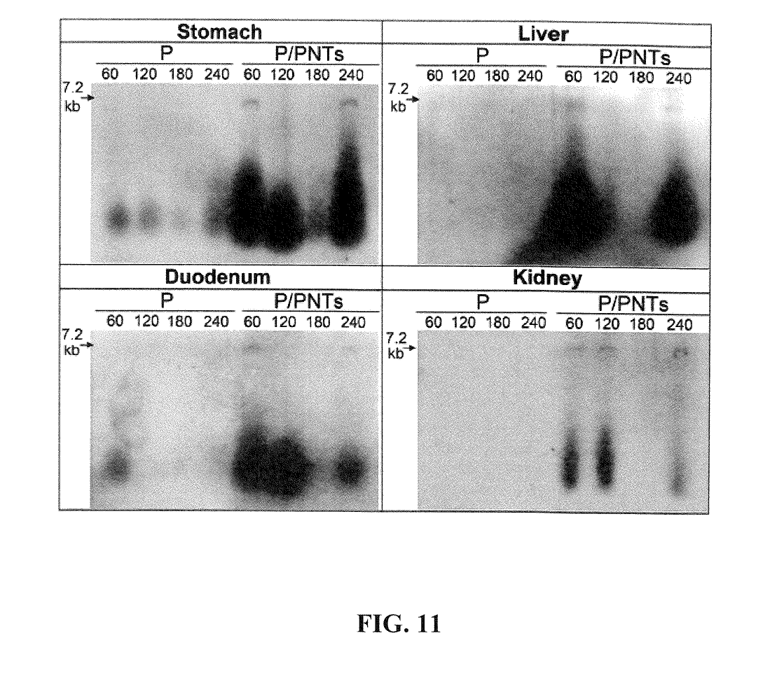

To further prove the existence of plasmid DNA in the stomach, duodenum, liver, and kidney those with significant lacZ gene expression, the presence of pCMV-lacZ plasmid DNA was analyzed by Southern blot analysis at indicated time after oral administration of naked pCMV-lacZ or pCMV-lacZ/PNTs prepared with TFA. With reference to FIG. 11 for southern blot analysis of the tissue DNAs from various tissues of mice with oral delivery of naked pCMV-lacZ or pCMV-lacZ formulated with peptide nanotube devices prepared with TFA, respectively, in accordance with an embodiment of the present invention, there was pCMV-lacZ DNA along with shorter fragmented DNAs in samples of stomach, duodenum, and liver at 1 h and in kidney at 1 and 2 h after oral administration of pCMV-lacZ/PNT formulation. However, only fragmented DNA was found in the samples of stomach and duodenum when mice receive naked plasmid DNA.

The mRNA of lacZ gene in four organs was also confirmed by RT-qPCR in samples from mice administered eight doses of pCMV-lacZ/PNTs prepared with TFA after 48 and 72 h of the first dose. AS shown in Table 5, the results revealed that lacZ mRNA was detected in samples from stomach, duodenum, liver, and kidney tissues at 48 and 72 h. However, no PCR product was detected when using cDNA from tissues of the plasmid DNA-treated control group.

TABLE 5
PNTs prepared with TFA

| | Time | | copies of mRNA/mg total RNA | | | |
|---|---|---|---|---|---|---|
| Gene | (h) | Formulation | stomach | duodenum | liver | kidney |
| lacZ | 48 | Plasmid/PNTs | 230400 ± 89197 | 254053 ± 92908 | 158400 ± 63308 | 182693 ± 58716 |
| (n = 6) | | Plasmid alone | ND | ND | ND | ND |
| | 72 | Plasmid/PNTs | 192227 ± 40983 | 245853 ± 35197 | 182960 ± 45265 | 167347 ± 413987 |
| | | Plasmid alone | ND | ND | ND | ND |
| hRluc | 48 | Plasmid/PNTs | 301760 ± 204845 | 369147 ± 46978 | 284000 ± 38276 | 286933 ± 100906 |
| (n = 3) | | Plasmid alone | ND | ND | ND | ND |
| | 72 | Plasmid/PNTs | 263733 ± 172038 | 297440 ± 117384 | 205173 ± 50769 | 176613 ± 52000 |
| | | Plasmid alone | ND | ND | ND | ND |

ND: non-determined

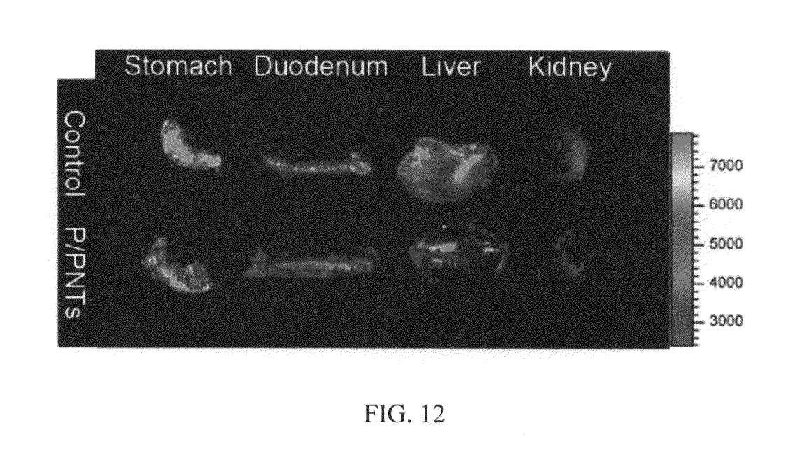

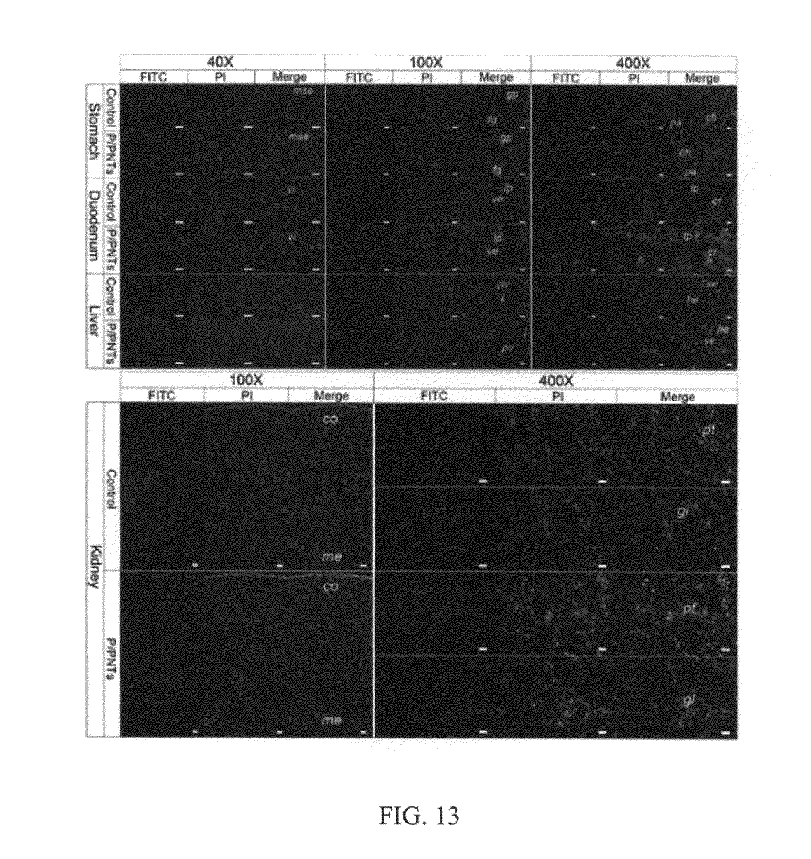

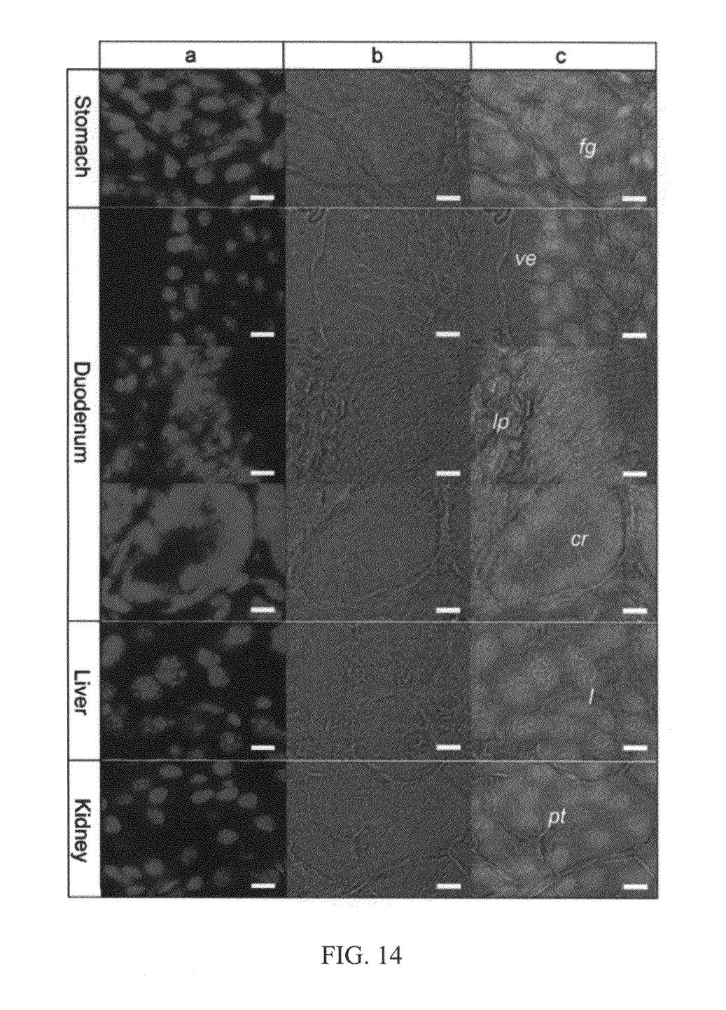

In addition, plasmid with the hRluc reporter was used to confirm the above results. The mRNA level, ex vivo bio-luminescence imaging, Renilla luciferase quantitative activity, and distribution in tissue sections of delivered DNA were analyzed. Similarly, hRluc mRNA was detected in stomach, duodenum, liver, and kidney tissues at 48 and 72 h after oral delivery of eight doses of pCMV-hRluc/PNTs prepared with TFA as shown in Table 5. With reference to FIG. 12 for an ex vivo bioluminescence view of the various tissues of mice with oral delivery of pCMV-hRluc formulated with peptide nanotube devices prepared with TFA in accordance with an embodiment of the present invention, results of ex vivo bioluminescence imaging revealed that the Renilla luciferase activity was observed in these four organs. The intensity variation of the signals was shown in different colors, as demonstrated by the color bar next to the figure. Referring to Table 6, the Renilla luciferase activity was significantly increased in the duodenum (59%) and kidney (40%) at 48 h and in the stomach (53%), duodenum (68%), and liver (43%) at 72 h after oral administration of eight doses of pCMV-hRluc/PNTs prepared with TFA (p<0.05). No significant Renilla luciferase activity was detected in all tissues after oral administration of plasmid DNA or PNTs prepared with TFA alone compared with that in the control group. With reference to FIG. 13 for immunohistological analysis of the various tissues of mice with oral delivery of pCMV-hRluc formulated with peptide nanotube devices prepared with TFA in accordance with an embodiment of the present invention, results of immunohistological analysis further confirmed that Renilla luciferase protein, which was shown in green in the figure, was found in the stomach, duodenum, liver, and kidney with nucleus stained with propidium iodide, shown in red. Wherein, mse denoted mucosa surface epithelium, gp denoted gastric pits, fg denoted fundus gland, pa denoted parietal cells, ch denoted chief cells, ye denoted villous epithelium, lp denoted lamina propria, cr denoted crypt cells, vi denoted duodenal villi, l denoted lobules, he denoted hepatocyte, se denoted sinusoidal endothelial cells, pv denoted portal vein, gl denoted glomerular, and pt denoted proximal tubular.

TABLE 6
PNTs prepared with TFA

| | Time | Renilla luciferase activity (RLU/mg protein) in tissues | | | |
|---|---|---|---|---|---|
| Formulation | (h) | stomach | duodenum | liver | kidney |
| pure control (n = 3) | 48 | 8817.62 ± 933.88 | 12635.08 ± 2901.22 | 7082.73 ± 1561.52 | 20008.25 ± 2122.86 |
| pCMV-hRluc alone (n = 3) | | 7325.65 ± 1310.89 | 11544.81 ± 2085.35 | 6818.67 ± 636.85 | 23422.88 ± 2122.86 |
| PNTs alone (n = 3) | | 7000.83 ± 2353.19 | 12406.62 ± 2796.98 | 7607.93 ± 1272.68 | 17069.47 ± 3923.51 |
| pCMV-hRluc/PNTs | | 9406.67 ± 1744.76 | 20116.74 ± 3070.11^{†} | 8508.11 ± 996.27 | 28096.46 ± 1526.47^{†} |
| (n = 3) | | | | | |
| pure control (n = 3) | 72 | 8320.74 ± 1643.37 | 11987.99 ± 757.96 | 7966.81 ± 742.48 | 19989.12 ± 1410.52 |
| pCMV-hRluc alone | | 9390.66 ± 1890.67 | 13993.65 ± 1339.97 | 7284.88 ± 1363.97 | 21215.97 ± 3016.47 |
| (n = 3) | | | | | |
| PNTs alone (n = 3) | | 6711.44 ± 2137.75 | 12703.84 ± 1722.50 | 7363.37 ± 1053.68 | 23357.28 ± 1906.80 |
| pCMV-hRluc/PNTs | | 12690.35 ± 789.86^{†} | 20118.91 ± 1254.21^{†} | 11376.05 ± 1261.74^{†} | 26304.52 ± 3439.30 |
| (n = 3) | | | | | |

^{†}significant difference (p < 0.05) compared with the same tissue of the control groups

TABLE 7
PNTs prepared with ethanol

| Formulation | | Renilla luciferase activity (RLU/mg protein) in tissues | | | |
|---|---|---|---|---|---|
| | Liver | Kidney | Brain | Testis | Stomach |
| pure control | 12355.91 ± 12106.29 | 7254.26 ± 6472.36 | 12350.16 ± 7405.81 | 9830.57 ± 12714.76 | 10549.26 ± 4538.88 |
| pCMV-hRluc | 23836.75 ± 26431.57 | 15731.68 ± 17426.89 | 26018.82 ± 16008.67 | 15728.21 ± 16743.12 | 22297.73 ± 20061.87 |
| alone | | | | | |
| pCMV-hRluc/ | 50159.51 ± 29806.00^{†} | 47591.38 ± 41989.29^{†} | 39097.52 ± 17027.48^{†} | 173291.46 ± 243597.79 | 76281.32 ± 77944.63^{†} |
| PNTs | | | | | |
| | Spinal cord | Spleen | Duodenum | Heart | Lung |
| pure control | 84719.38 ± 40577.59 | 83216.13 ± 78052.17 | 76288.41 ± 48558.50 | 21174.86 ± 10384.42 | 30445.72 ± 33025.95 |
| pCMV-hRluc | 85050.83 ± 31043.57 | 187983.70 ± 98714.69 | 47464.96 ± 28990.08 | 37182.68 ± 23284.50 | 67273.30 ± 72307.44 |
| alone | | | | | |
| pCMV-hRluc/ | 99024.85 ± 45745.23 | 212912.22 = 204483.69 | 129729.13 ± 84222.96^{†} | 51519.02 ± 37352.09 | 267300.70 ± 204389.55^{†} |
| PNTs | | | | | |

^{†}significant difference (p < 0.05) compared with the same tissue of the control groups

To trace the presence of PNTs prepared with TFA in tissue sections of the stomach, duodenum, liver, and kidney, mice were orally administered with thioflavin T (ThT) pre-stained PNTs prepared with TFA. With reference to FIG. 14 for ThT image (green) merged with DAPI image (blue) (a), bright field image (b), and ThT image merged with DAPI image and bright field image (c), respectively, of histological analysis of the various tissues of mice with oral delivery of peptide nanotube devices prepared with TFA in accordance with an embodiment of the present invention, the results revealed the smaller PNTs prepared with TFA (<5 μm in length), which was green in the figure, were found in the sections of stomach, duodenum, liver, and kidney, indicating the presence of degraded PNTs prepared with TFA in these tissues. Wherein, fg denoted fundus gland, ye denoted villous epithelium, lp denoted lamina propria, cr denoted crypt cells, vi denoted duodenal villi, l denoted lobules, and pt denoted proximal tubular.

After 48 h of the first oral pCMV-hRluc/PNTs dose, wherein the PNTs of which is prepared with ethanol, mice were sacrificed and the Renilla luciferase quantitative activity in various organs, including duodenum, testis, kidney, stomach, heart, liver, brain, lung, spinal cord and spleen were evaluated. Referring to Table 7, results showed that the Renilla luciferase activity significantly increased in liver, kidney, brain, stomach, duodenum, and lung at 48 h after oral administration of the first dose of pCMV-hRluc/PNTs (p<0.05).

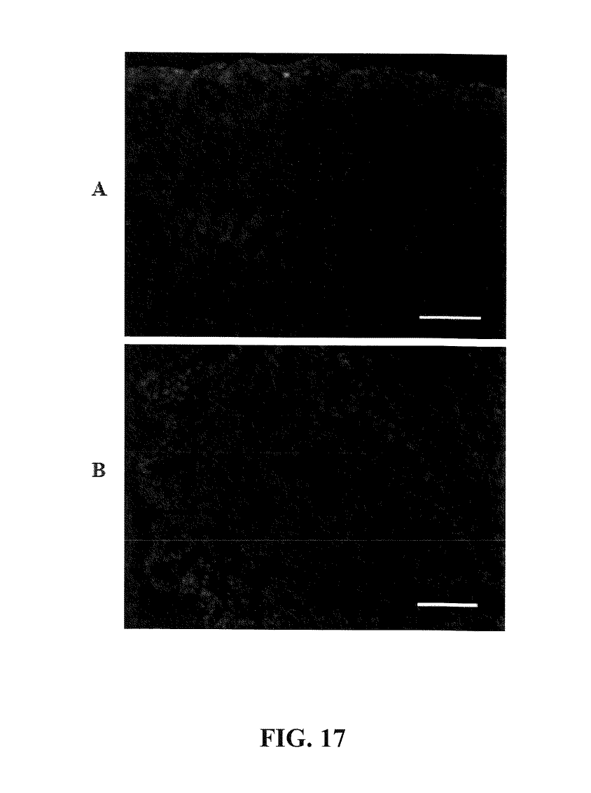

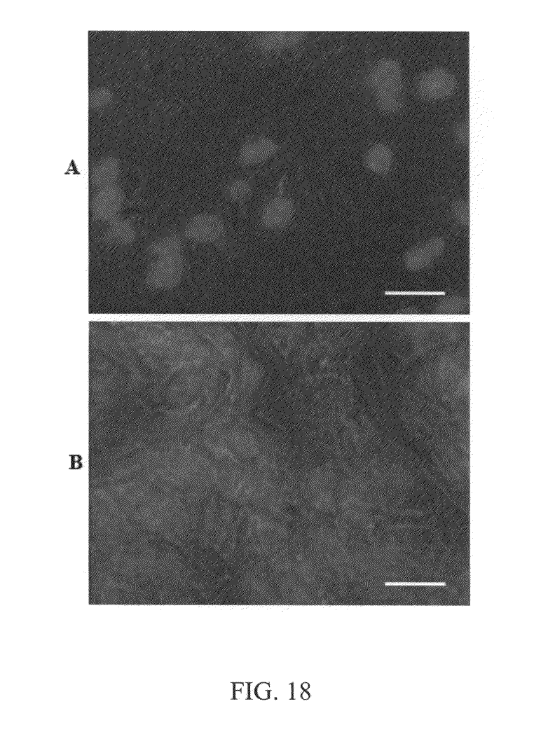

With reference to parts A and B of FIG. 17 for immunohistological analysis of liver tissues and lung tissues, respectively, of mice with oral delivery of pCMV-hRluc formulated with peptide nanotube devices prepared with ethanol in accordance with an embodiment of the present invention, results of histological analysis showed that hRluc activity was found in liver and lung, detected by anti-Renilla antibody, as shown in green. On the other hand, with reference to parts A and B of FIG. 18 for histological analysis of the brain tissues and lung tissues of mice with orally delivery of ThT pre-stained peptide nanotube devices prepared with ethanol in accordance with an embodiment of the present invention, to trace the presence of PNTs in tissue sections of brain and lung, mice were orally administrated with ThT pre-stained PNTs. Referring to parts A and B of FIG. 18, the results revealed that the PNTs prepared with ethanol, shown in green, were found in the sections of brain and lung area, indicating the presence of PNTs in these tissues.

### Topically Eye Drop Gene Transfer In Vivo

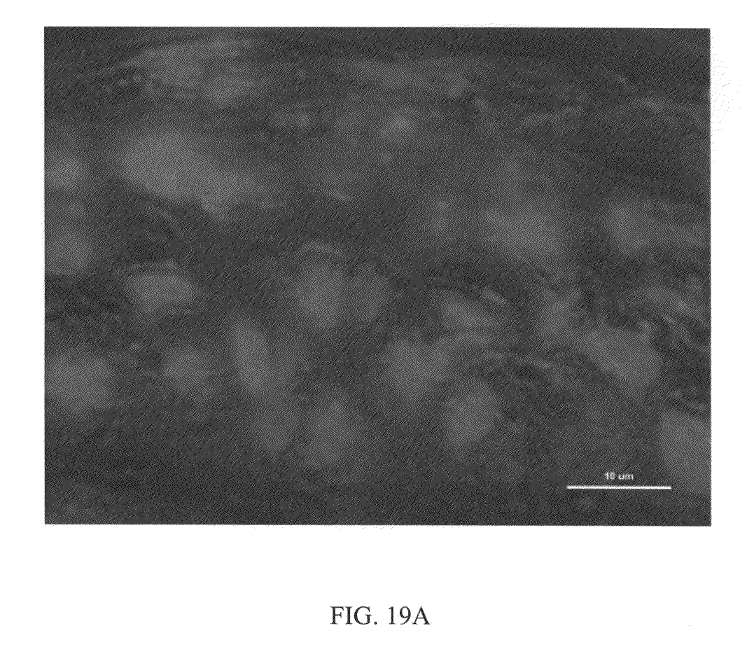

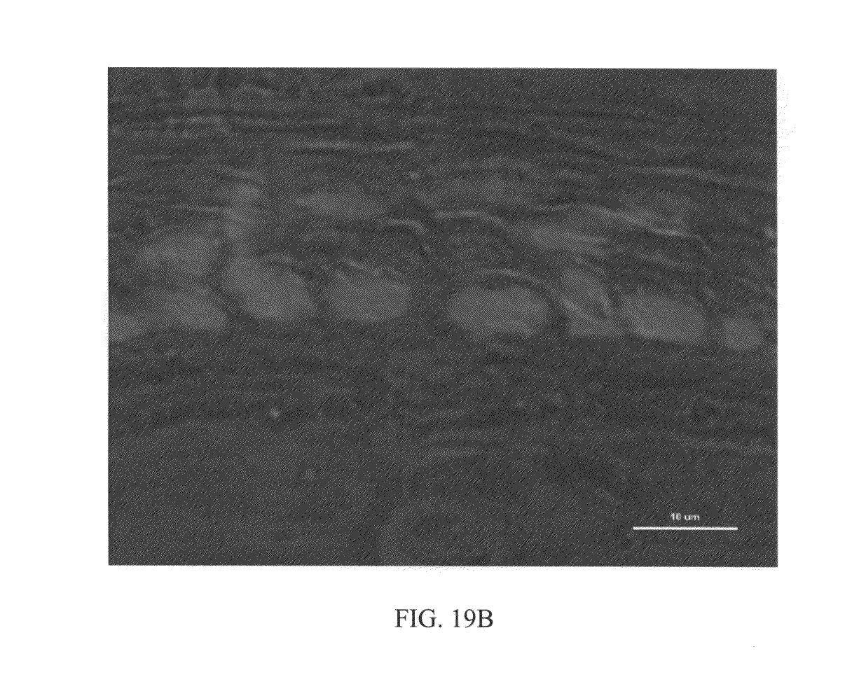

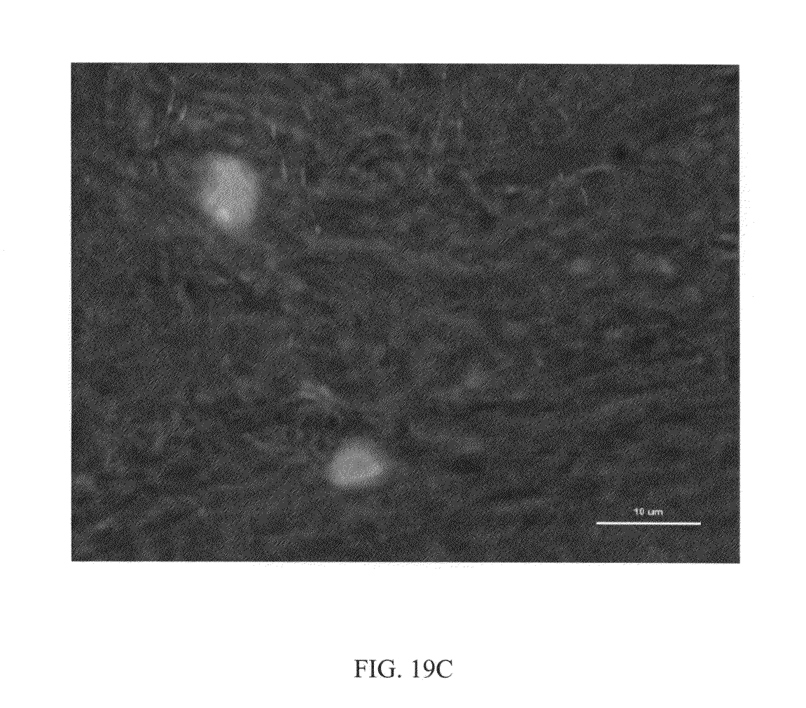

With reference to parts A and B of FIG. 19 for ThT images merged with DAPI image and bright field image of histological analysis of the epithelial layers of the cornea tissues, and part C of FIG. 19 for a ThT image merged with DAPI image and bright field image of histological analysis of the stroma layers of the cornea tissues of mice with topically eye drop delivery of ThT pre-stained peptide nanotube devices prepared with ethanol in accordance with an embodiment of the present invention. After 2 hours of the first topically eye drop of ThT pre-stained PNTs dose, cornea tissues of mice were obtained and the PNTs observed in various layer of cornea, including epithelial area and stroma area. Referring to parts A-C of FIG. 19, the results revealed that the smaller PNTs, shown in green, were also found in the layer of epithelial area and stroma area of the cornea tissues, indicating the presence of PNTs in these tissues.

### CONCLUSIONS

In summary, the association of plasmid DNA with cyclo-(D-Trp-Tyr) peptide PNTs enhanced the duodenal permeability of plasmid DNA in vitro. The in vivo study revealed that the β-Gal activity and Renilla luciferase were significantly increased after the first dose of plasmid/PNT formulation by oral administration. The organs with increased lacZ expression, including the duodenum, stomach, liver, and kidney, were confirmed by the presence of DNA using both Southern blot analysis and TM-rhodamine-labeled DNA tracing. Both lacZ and hRluc mRNAs were detected in these four organs at 48 and 72 h after the first dose of oral delivery. These results implicate the potential application of cyclo-(D-Trp-Tyr) peptide PNTs as a nano-vector for oral gene delivery to the duodenum, stomach, liver, brain, lung, and kidney as well as in cornea by topical eye drop delivery.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

### REFERENCES

(1) Na, K.; Jung, J.; Lee, J.; Hyun, J. Thermoresponsive Pore Structure of Biopolymer Microspheres for a Smart Drug Carrier. Langmuir 2010, 26, 11165-11169.
(2) Na, K.; Lee, S. A.; Jung, S. H.; Hyun, J.; Shin, B. C. Elastin-Like Polypeptide Modified Liposomes for Enhancing Cellular Uptake into Tumor Cells. Colloids Surf., B 2012, 91, 130-136.
(3) Tong, Y. C.; Yu, T. Y.; Chang, S. F.; Liaw, J. Nanopolymeric Micelle Effect on the Transdermal Permeability, the Bioavailability and Gene Expression of Plasmid. Mol. Pharmaceutics 2012, 9, 111-20.
(4) Chen, C. C.; Liu, Y. C.; Wu, C. H.; Yeh, C. C.; Su, M. T.; Wu, Y. C. Preparation of Fluorescent Silica Nanotubes and Their Application in Gene Delivery. Adv. Mater. 2005, 17, 404-407.
(5) Huang, X.; Teng, X.; Chen, D.; Tang, F.; He, J. The Effect of the Shape of Mesoporous Silica Nanoparticles on Cellular Uptake and Cell Function. Biomaterials 2010, 31, 438-448.
(6) O'Connell, M. J.; Boul, P.; Ericson, L. M.; Huffman, C.; Wang, Y.; Haroz, E.; Kuper, C.; Tour, J.; Ausman, K. D.; Smalley, R. E. Reversible Water-Solubilization of Single-Walled Carbon Nanotubes by Polymer Wrapping. Chem. Phys. Lett. 2001, 342, 265-271.
(7) Chin, S. F.; Baughman, R. H.; Dalton, A. B.; Dieckmann, G. R.; Draper, R. K.; Mikoryak, C.; Musselman, I. H.; Poenitzsch, V. Z.; Xie, H.; Pantano, P. Amphiphilic Helical Peptide Enhances the Uptake of Single-Walled Carbon Nanotubes by Living Cells. Exp. Biol. Med. 2007, 232, 1236-1244.
(8) Cui, D.; Tian, F.; Ozkan, C. S.; Wang, M.; Gao, H. Effect of Single Wall Carbon Nanotubes on Human HEK293 Cells. Toxicol. Lett. 2005, 155, 73-85.
(9) Lam, C. W.; James, J. T.; McCluskey, R.; Hunter, R. L. Pulmonary Toxicity of Single-Wall Carbon Nanotubes in Mice 7 and 90 Days after Intractracheal Instillation. Toxicol. Sci. 2004, 77, 126-134. 928
(10) Porter, D. W.; Hubbs, A. F.; Mercer, R. R.; Wu, N.; Wolfarth, M. G.; Sriram, K.; Leonard, S.; Battelli, L.; Schwegler-Berry, D.; Friend, S.; Andrew, M.; Chen, B. T.; Tsuruoka, S.; Endo, M.; Castranova, V. Mouse Pulmonary Dose and Time Course Responses Induced by Exposure to Multi-Walled Carbon Nanotubes. Toxicology 2010, 269, 136-147.
(11) Ji, Z.; Zhang, D.; Li, L.; Shen, X.; Deng, X.; Dong, L.; Wu, M.; Liu, Y. The Hepatotoxicity of Multi-Walled Carbon Nanotubes in Mice. Nanotechnology 2009, 20, 445101.
(12) Kam, N. W. S.; Jessop, T. C.; Wender, P. A.; Dai, H. Nanotube Molecular Transporters: Internalization of Carbon Nanotube-Protein Conjugates into Mammalian Cells. J. Am. Chem. Soc. 2004, 126, 6850-6851.
(13) Kang, B.; Chang, S.; Dai, Y.; Yu, D.; Chen, D. Cell Response to Carbon Nanotubes: Size-Dependent Intracellular Uptake Mechanism and Subcellular Fate. Small 2010, 6, 2362-2366.
(14) Champion, J. A.; Mitragotri, S. Role of Target Geometry in Phagocytosis. Proc. Natl. Acad. Sci. U.S.A. 2006, 103, 4930-4934.
(15) Qiu, Y.; Liu, Y.; Wang, L.; Xu, L.; Bai, R.; Ji, Y.; Wu, X.; Zhao, Y.; Li, Y.; Chen, C. Surface Chemistry and Aspect Ratio Mediated Cellular Uptake of Au Nanorods. Biomaterials 2010, 31, 7606-7619.
(16) Bhirde, A. A.; Patel, S.; Sousa, A. A.; Patel, V.; Molinolo, A. A.; Ji, Y.; Leapman, R. D.; Gutkind, J. S.; Rusling, J. F. Distribution and Clearance of PEG-Single-Walled Carbon Nanotube Cancer Drug Delivery Vehicles in Mice. Nanomedicine 2010, 5, 1535-1546.
(17) Liaw, J.; Chang, S. F.; Hsiao, F. C. In Vivo Gene Delivery into Ocular Tissues by Eye Drops of Poly(ethylene oxide)-Poly(propylene oxide)-Poly(ethylene oxide) (PEO-PPO-PEO) Polymeric Micelles. Gene Ther. 2001, 8, 999-1004.
(18) Xiao, J.; Wei, X.; Wang, Y.; Liu, C. Fluorescence Resonance Energy-Transfer Affects the Determination of the Affinity between Ligand and Proteins Obtained by Fluorescence Quenching Method. Spectrochim. Acta A 2009, 74, 977-982.
(19) Mishra, B.; Barik, A.; Priyadarsini, K. I.; Mohan, H. Fluorescence Spectroscopic Studies on Binding of a Flavonoid Antioxidant Quercetin to Serum Albumins. J. Chem. Sci. 2005, 117, 641-647.

1-12. (canceled)

13. A method for manufacturing a peptide nanotube (PNT) device, comprising:
dissolving a cyclo-(D-Trp-Tyr) peptide powder in a solvent to be a solution in a container;
incubating the solution at a predetermined temperature for a predetermined time for the solvent to evaporate to obtain peptide nanotubes formed of cyclo-(D-Trp-Tyr) peptide; and
mixing the peptide nanotube with a matrix including biomolecules to obtain the PNT device;
wherein the solvent is 0.1 to 5% (v/v) trifluoroacetic acid aqueous solution or 1 to 100% (v/v) ethanol aqueous solution.

14-15. (canceled)

16. The method of claim 13, wherein a volume of the trifluoroacetic acid is 0.015 to 0.75 mL.

17. The method of claim 16, wherein a weight of the cyclo-(D-Trp-Tyr) peptide powder is 0.1 to 10 mg.

18. The method of claim 13, wherein after dissolving the cyclo-(D-Trp-Tyr) peptide powder in the solvent, the method further comprises the step of adding double distilled water to the solution at the predetermined temperature.

19. The method of claim 18, wherein the predetermined temperature is 0-25° C.

20. The method of claim 19, wherein the predetermined time is 10-72 hours.

21-22. (canceled)

23. The method of claim 13, wherein a volume of the ethanol is 0.1 to 10 mL.

24. The method of claim 23, wherein a weight of the cyclo-(D-Trp-Tyr) peptide powder is 0.1 to 10 mg.

25. The method of claim 13, wherein the predetermined temperature is 0-25° C.

26. The method of claim 25, wherein the predetermined time is 1-48 hours.

27. The method of claim 13, wherein the biomolecules comprise peptides, proteins, nucleic acids and drugs.

28. The method of claim 27, wherein the nucleic acids comprise DNA, shRNA and siRNA.

29. The method of claim 28, wherein a concentration of the DNA is 0.01-0.3 μg/μL.

30. The method of claim 29, wherein a concentration of the peptide nanotubes is 0.05-5% (w/v).

31. The method of claim 28, wherein a release rate of the PNT device to release DNA is about 1×10¹⁰ to 5×10¹¹ copies DNA/t^{1/2}.

32. The method of claim 13, wherein a width of the PNTs is 10 to 800 nm.

33. The method of claim 13, wherein a length of the PNTs is 0.1 to 20 μm.

34. The method of claim 13, wherein the PNTs are bundled or aggregated nanotubes.

35. The method of claim 13, wherein a zeta potential of the PNT device is −10 to 10 mV.


SEQUENCE LISTING (4 sequences)

SEQ ID NO: 1 - DNA, 22 bp, Artificial Sequence
  qPCR forward primer for B-Gal
ctacaccaacgtaacctatccc

SEQ ID NO: 2 - DNA, 22 bp, Artificial Sequence
  qPCR reverse primer for B-Gal
ttctccggcgcgtaaaaatgcg

SEQ ID NO: 3 - DNA, 22 bp, Artificial Sequence
  qPCR forward primer for Renilla luciferase
tccctgatctgatcggaatggg

SEQ ID NO: 4 - DNA, 22 bp, Artificial Sequence
  qPCR reverse primer for Renilla luciferase
cttggtgctcgtaggagtagtg